SEQUENCE LISTING (82 sequences)

SEQ ID NO: 1 - protein, 140 aa, synthetic construct
  REGION 1..140: note = Human IL-5 amino acid sequence with his tag (rhIL-5-his)
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIESHHHHHH

SEQ ID NO: 2 - protein, 140 aa, synthetic construct
  REGION 1..140: note = Cynomolgus monkey IL-5 amino acid sequence with his tag
MRMLLHLSLLALGAAYVYAIPTEIPASALVKETLALLSTHRTLLIANETLRIPVPVHKNHQLCTEEIFQGIGTLESQTVQGGTVERLFKNLSLIKKYIGGQKKKCGEERRRVNQFLDYLQEFLGVMNTEWIIESHHHHHH

SEQ ID NO: 3 - protein, 119 aa, synthetic construct
  REGION 1..119: note = Mouse IL-5 amino acid sequence with his tag
MEIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEGHHHHHH

SEQ ID NO: 4 - protein, 119 aa, synthetic construct
  REGION 1..119: note = Rat IL-5 amino acid sequence with his tag
MEIPMSTVVKETLIQLSTHRALLTSNETMRLPVPTHKNHQLCIGEIFQGLDILKNQTVRGGTVEILFQNLSLIKKYIDGQKEKCGEERRKTRHFLDYLQEFLGVMSTEWAMEVHHHHHH

SEQ ID NO: 5 - protein, 560 aa, synthetic construct
  REGION 1..560: note = human IL-5 alpha fused to human Fc fragment
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 118 aa, synthetic construct
  REGION 1..118: note = mAb1705 murine heavy chain variable region sequence
EVQLVESGGGLVQPGRSLKLSCTASGFTFSHYYMAWVRQAPKKGLEWVTSISYEGDITYYGDSVKGRFTISRDNAKSTLYLQMNSLRSEDTATYYCASQTLRESFDYWGQGVMVTVSS

SEQ ID NO: 7 - protein, 107 aa, synthetic construct
  REGION 1..107: note = mAb1705 murine light chain variable region sequence
DIQMTQSPSSMSVSLGDRVTITCRASQDIANYLSWYQQKIARSPKLVIYGTSNLEVGVPSRFSGSRSGSDYSLTINTLESEDTGIYFCLQDKEFPRTFGGGTRLELK

SEQ ID NO: 8 - protein, 118 aa, synthetic construct
  REGION 1..118: note = mAb1706 murine heavy chain variable region sequence
EVQLVESGGGLVQPGRSLKLSCAASGFTFSHYYMAWVRQAPKKGLEWVTSINYEGNSAYYGDSVKGRFTISRDNAKSTLYLQMDSLRSEDTATYYCATETLRESLDYWGQGVMVTVSS

SEQ ID NO: 9 - protein, 107 aa, synthetic construct
  REGION 1..107: note = mAb1706 murine light chain variable region sequence
DIQMTQSPSSMSVSLGDRVTITCRASQDIGNYLSWYQQKLGKSPKLMIHSASNLEVGVPSRFSGSRSGSDYSLTINTLESEDPGIYFCLQHKQFPRTFGGGTKLELK

SEQ ID NO: 10 - protein, 119 aa, synthetic construct
  REGION 1..119: note = mAb1780 murine heavy chain variable region sequence
QVKLLQSGAALVKPGDSVKMSCKASDYTFTEYLIHWVKQSQGRSLEWIGYINPYSGGTVYNEKFKSKATLTVDKFSSTAYMEFRRLTFEDSAIYYCARDGGYSDPLDYWGQGVMVTVSS

SEQ ID NO: 11 - protein, 106 aa, synthetic construct
  REGION 1..106: note = mAb1780 murine light chain variable region sequence
DTVLTQSPALAVSPGERVSISCRASEGLTSYMHWYQQKPGQQPKLLIYKASNLASGVPARFSGSGSGTDFTLTIDPVEADDAATYFCQQNWNDPWTFGGGTKLELK

SEQ ID NO: 12 - protein, 116 aa, synthetic construct
  REGION 1..116: note = mAb1773 murine heavy chain variable region sequence
EVQLQQSLAELVRPGASVTLSCTASGFNIKNTYIHWVKQRPEQGLEWIGRIDPANGDTKHGPKFQGKATITADTSSNTAYLQFSSLTSEDTAIYYCFRYGIYPDHWGQGTPLTVSS

SEQ ID NO: 13 - protein, 106 aa, synthetic construct
  REGION 1..106: note = mAb1773 murine light chain variable region sequence
QIVLTQSPALMSASPGEKVTMTCSASSSVNYIYWYQQKPRSSPKPWIYLTATLASGVPARFSGSGSGTSFSLTISRMEAEDAATYYCQQWNSYPYTFGGGTKLEIE

SEQ ID NO: 14 - protein, 120 aa, synthetic construct
  REGION 1..120: note = mAb1779 murine heavy chain variable region sequence
QVKLLQSGAALVKPGDSVKMSCKASGYTFTDYIIHWVKQSHGKSLEWIGYFNPNSGGSNYNENFKRKATLTADKSSSTAYLEFSRVTSEDSAIYYCGRRIAWDHWYFDFWGPGTMVTVSS

SEQ ID NO: 15 - protein, 107 aa, synthetic construct
  REGION 1..107: note = mAb1779 murine light chain variable region sequence
DIQMTQSPASLSASLGETVSIECLASEGISNDVAWYQQKSGRSPQLLVYAASRLQDGVPSRFSGSGSGTRYFFKISGMQPEDEADYFCQQGYKTPLTFGSGTKLEIK

SEQ ID NO: 16 - protein, 5 aa, synthetic construct
  REGION 1..5: note = mAb1705 HCDR1
HYYMA

SEQ ID NO: 17 - protein, 17 aa, synthetic construct
  REGION 1..17: note = mAb1705 HCDR2
SISYEGDITYYGDSVKG

SEQ ID NO: 18 - protein, 9 aa, synthetic construct
  REGION 1..9: note = mAb1705 HCDR3
QTLRESFDY

SEQ ID NO: 19 - protein, 11 aa, synthetic construct
  REGION 1..11: note = mAb1705 LCDR1
RASQDIANYLS

SEQ ID NO: 20 - protein, 7 aa, synthetic construct
  REGION 1..7: note = mAb1705 LCDR2
GTSNLEV

SEQ ID NO: 21 - protein, 9 aa, synthetic construct
  REGION 1..9: note = mAb1705 LCDR3
LQDKEFPRT

SEQ ID NO: 22 - protein, 5 aa, synthetic construct
  REGION 1..5: note = mAb1706 HCDR1
HYYMA

SEQ ID NO: 23 - protein, 17 aa, synthetic construct
  REGION 1..17: note = mAb1706 HCDR2
SINYEGNSAYYGDSVKG

SEQ ID NO: 24 - protein, 9 aa, synthetic construct
  REGION 1..9: note = mAb1706 HCDR3
ETLRESLDY

SEQ ID NO: 25 - protein, 11 aa, synthetic construct
  REGION 1..11: note = mAb1706 LCDR1
RASQDIGNYLS

SEQ ID NO: 26 - protein, 7 aa, synthetic construct
  REGION 1..7: note = mAb1706 LCDR2
SASNLEV

SEQ ID NO: 27 - protein, 9 aa, synthetic construct
  REGION 1..9: note = mAb1706 LCDR3
LQHKQFPRT

SEQ ID NO: 28 - protein, 5 aa, synthetic construct
  REGION 1..5: note = mAb1780 HCDR1
EYLIH

SEQ ID NO: 29 - protein, 17 aa, synthetic construct
  REGION 1..17: note = mAb1780 HCDR2
YINPYSGGTVYNEKFKS

SEQ ID NO: 30 - protein, 10 aa, synthetic construct
  REGION 1..10: note = mAb1780 HCDR3
DGGYSDPLDY

SEQ ID NO: 31 - protein, 11 aa, synthetic construct
  REGION 1..11: note = mAb1780 LCDR1
RASEGLTSYMH

SEQ ID NO: 32 - protein, 7 aa, synthetic construct
  REGION 1..7: note = mAb1780 LCDR2
KASNLAS

SEQ ID NO: 33 - protein, 9 aa, synthetic construct
  REGION 1..9: note = mAb1780 LCDR3
QQNWNDPWT

SEQ ID NO: 34 - protein, 5 aa, synthetic construct
  REGION 1..5: note = mAb1773 HCDR1
NTYIH

SEQ ID NO: 35 - protein, 17 aa, synthetic construct
  REGION 1..17: note = mAb1773 HCDR2
RIDPANGDTKHGPKFQG

SEQ ID NO: 36 - protein, 7 aa, synthetic construct
  REGION 1..7: note = mAb1773 HCDR3
YGIYPDH

SEQ ID NO: 37 - protein, 10 aa, synthetic construct
  REGION 1..10: note = mAb1773 LCDR1
SASSSVNYIY

SEQ ID NO: 38 - protein, 7 aa, synthetic construct
  REGION 1..7: note = mAb1773 LCDR2
LTATLAS

SEQ ID NO: 39 - protein, 9 aa, synthetic construct
  REGION 1..9: note = mAb1773 LCDR3
QQWNSYPYT

SEQ ID NO: 40 - protein, 5 aa, synthetic construct
  REGION 1..5: note = mAb1779 HCDR1
DYIIH

SEQ ID NO: 41 - protein, 17 aa, synthetic construct
  REGION 1..17: note = mAb1779 HCDR2
YFNPNSGGSNYNENFKR

SEQ ID NO: 42 - protein, 11 aa, synthetic construct
  REGION 1..11: note = mAb1779 HCDR3
RIAWDHWYFDF

SEQ ID NO: 43 - protein, 11 aa, synthetic construct
  REGION 1..11: note = mAb1779 LCDR1
LASEGISNDVA

SEQ ID NO: 44 - protein, 7 aa, synthetic construct
  REGION 1..7: note = mAb1779 LCDR2
AASRLQD

SEQ ID NO: 45 - protein, 9 aa, synthetic construct
  REGION 1..9: note = mAb1779 LCDR3
QQGYKTPLT

SEQ ID NO: 46 - protein, 107 aa, synthetic construct
  REGION 1..107: note = h1705_VL.1
DIQMTQSPSSVSASVGDRVTITCRASQDIANYLSWYQQKPGKAPKLLIYGTSNLEVGVPSRFSGSGSGTDFTLTISSLQPEDFATYYCLQDKEFPRTFGGGTKVEIK

SEQ ID NO: 47 - protein, 107 aa, synthetic construct
  REGION 1..107: note = h1705_VL.1A
DIQMTQSPSSVSASVGDRVTITCRASQDIANYLSWYQQKPGKSPKLLIYGTSNLEVGVPSRFSGSRSGTDFTLTISSLQPEDFATYYCLQDKEFPRTFGGGTKVEIK

SEQ ID NO: 48 - protein, 107 aa, synthetic construct
  REGION 1..107: note = h1705_VL.1B
DIQMTQSPSSVSASVGDRVTITCRASQDIANYLSWYQQKPGKSPKLVIYGTSNLEVGVPSRFSGSRSGSDYTLTISSLQPEDFATYFCLQDKEFPRTFGGGTKVEIK

SEQ ID NO: 49 - protein, 118 aa, synthetic construct
  REGION 1..118: note = h1705_VH.1
EVQLVESGGGLVQPGGSLRLSCAASGFTFSHYYMAWVRQAPGKGLEWVSSISYEGDITYYGDSVKGRFTISRDNSKNTLYLQMNSLRAEDTAVYYCAKQTLRESFDYWGQGTLVTVSS

SEQ ID NO: 50 - protein, 118 aa, synthetic construct
  REGION 1..118: note = h1705_VH.1A
EVQLVESGGGLVQPGGSLRLSCAASGFTFSHYYMAWVRQAPGKGLEWVSSISYEGDITYYGDSVKGRFTISRDNSKNTLYLQMNSLRAEDTAVYYCASQTLRESFDYWGQGTLVTVSS

SEQ ID NO: 51 - protein, 118 aa, synthetic construct
  REGION 1..118: note = h1705_VH.1B
EVQLVESGGGLVQPGGSLRLSCAASGFTFSHYYMAWVRQAPGKGLEWVTSISYEGDITYYGDSVKGRFTISRDNSKNTLYLQMNSLRAEDTATYYCASQTLRESFDYWGQGTLVTVSS

SEQ ID NO: 52 - protein, 330 aa, synthetic construct
  REGION 1..330: note = Heavy chain IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 107 aa, synthetic construct [sequence omitted]

SEQ ID NO: 54 - protein, 107 aa, synthetic construct
  REGION 1..107: note = h1706_VL.1
DIQMTQSPSSVSASVGDRVTITCRASQDIGNYLSWYQQKPGKAPKLLIYSASNLEVGVPSRFSGSGSGTDFTLTISSLQPEDFATYYCLQHKQFPRTFGGGTKVEIK

SEQ ID NO: 55 - protein, 107 aa, synthetic construct
  REGION 1..107: note = h1706_VL.1A
DIQMTQSPSSVSASVGDRVTITCRASQDIGNYLSWYQQKPGKSPKLLIYSASNLEVGVPSRFSGSGSGTDFTLTISSLQPEDFATYYCLQHKQFPRTFGGGTKVEIK

SEQ ID NO: 56 - protein, 107 aa, synthetic construct
  REGION 1..107: note = h1706_VL.1B
DIQMTQSPSSVSASVGDRVTITCRASQDIGNYLSWYQQKPGKSPKLMIYSASNLEVGVPSRFSGSGSGTDYTLTISSLQPEDFATYFCLQHKQFPRTFGGGTKVEIK

SEQ ID NO: 57 - protein, 118 aa, synthetic construct
  REGION 1..118: note = h1706_VH.1
EVQLVESGGGLVQPGGSLRLSCAASGFTFSHYYMAWVRQAPGKGLEWVSSINYEGNSAYYGDSVKGRFTISRDNSKNTLYLQMNSLRAEDTAVYYCAKETLRESLDYWGQGTMVTVSS

SEQ ID NO: 58 - protein, 118 aa, synthetic construct
  REGION 1..118: note = h1706_VH.1A
EVQLVESGGGLVQPGGSLRLSCAASGFTFSHYYMAWVRQAPGKGLEWVSSINYEGNSAYYGDSVKGRFTISRDNSKNTLYLQMNSLRAEDTAVYYCATETLRESLDYWGQGTMVTVSS

SEQ ID NO: 59 - protein, 118 aa, synthetic construct
  REGION 1..118: note = h1706_VH.1B
EVQLVESGGGLVQPGGSLRLSCAASGFTFSHYYMAWVRQAPGKGLEWVTSINYEGNSAYYGDSVKGRFTISRDNSKNTLYLQMNSLRAEDTATYYCATETLRESLDYWGQGTMVTVSS

SEQ ID NO: 60 - protein, 107 aa, synthetic construct
  REGION 1..107: note = h1780_VL.1
EIVLTQSPATLSLSPGERATLSCRASEGLTSYMHWYQQKPGQAPRLLIYKASNLASGIPARFSGSGSGTDFTLTISSLEPEDFAVYYCQQNWNDPWTFGGGTKVEIK

SEQ ID NO: 61 - protein, 107 aa, synthetic construct
  REGION 1..107: note = h1780_VL.1A
DTVLTQSPATLSLSPGERATLSCRASEGLTSYMHWYQQKPGQAPRLLIYKASNLASGIPARFSGSGSGTDFTLTISSLEPEDFAVYYCQQNWNDPWTFGGGTKVEIK

SEQ ID NO: 62 - protein, 107 aa, synthetic construct
  REGION 1..107: note = h1780_VL.1B
DTVLTQSPATLSLSPGERATLSCRASEGLTSYMHWYQQKPGQAPRLLIYKASNLASGVPARFSGSGSGTDFTLTISSLEPEDFATYFCQQNWNDPWTFGGGTKVEIK

SEQ ID NO: 63 - protein, 119 aa, synthetic construct
  REGION 1..119: note = h1780_VH.1
EVQLVQSGAEVKKPGASVKVSCKASGYTFTEYLIHWVRQAPGQGLEWMGYINPYSGGTVYNEKFKSRVTMTRDTSISTAYMELSRLRSDDTAVYYCARDGGYSDPLDYWGQGTMVTVSS

SEQ ID NO: 64 - protein, 119 aa, synthetic construct
  REGION 1..119: note = h1780_VH.1A
EVQLVQSGAEVKKPGASVKVSCKASGYTFTEYLIHWVRQAPGQGLEWMGYINPYSGGTVYNEKFKSRVTLTVDKSISTAYMELSRLRSDDTAVYYCARDGGYSDPLDYWGQGTMVTVSS

SEQ ID NO: 65 - protein, 119 aa, synthetic construct
  REGION 1..119: note = h1780_VH.1B
EVQLVQSGAEVKKPGASVKVSCKASGYTFTEYLIHWVRQAPGQGLEWIGYINPYSGGTVYNEKFKSRATLTVDKSISTAYMEFSRLRSDDTAVYYCARDGGYSDPLDYWGQGTMVTVSS

SEQ ID NO: 66 - protein, 119 aa, synthetic construct
  REGION 1..119: note = h1780_VH.1C
EVQLVQSGAEVKKPGASVKVSCKASGYTFTEYLIHWVKQAPGQGLEWIGYINPYSGGTVYNEKFKSKATLTVDKSISTAYMEFSRLRSDDTAVYYCARDGGYSDPLDYWGQGTMVTVSS

SEQ ID NO: 67 - protein, 106 aa, synthetic construct
  REGION 1..106: note = h1773_VL.1
DIQMTQSPSSLSASVGDRVTITCSASSSVNYIYWYQQKPGKAPKLLIYLTATLASGVPSRFSGSGSGTDFTLTISSLQPEDFATYYCQQWNSYPYTFGGGTKVEIK

SEQ ID NO: 68 - protein, 106 aa, synthetic construct
  REGION 1..106: note = h1773_VL.1A
DIQLTQSPSSLSASVGDRVTITCSASSSVNYIYWYQQKPGKSPKPWIYLTATLASGVPSRFSGSGSGTDFTLTISSLQPEDFATYYCQQWNSYPYTFGGGTKVEIK

SEQ ID NO: 69 - protein, 116 aa, synthetic construct
  REGION 1..116: note = h1773_VH.1
EVQLVESGGGLVQPGGSLKLSCAASGFTFSNTYIHWVRQASGKGLEWVGRIDPAVGDTKHGPKFQGRFTISRDDSKNTAYLQMNSLKTEDTAVYYCTRYGIYPDHWGQGTLVTVSS

SEQ ID NO: 70 - protein, 116 aa, synthetic construct
  REGION 1..116: note = h1773_VH.1A
EVQLVESGGGLVQPGGSLKLSCAASGFTISNTYIHWVRQASGKGLEWVGRIDPAVGDTKHGPKFQGRFTISADDSKNTAYLQMNSLKTEDTAVYYCFRYGIYPDHWGQGTLVTVSS

SEQ ID NO: 71 - protein, 116 aa, synthetic construct
  REGION 1..116: note = h1773_VH.1B
EVQLVESGGGLVQPGGSLKLSCAASGFTISNTYIHWVKQASGKGLEWIGRIDPAVGDTKHGPKFQGRFTISADDSKNTAYLQMNSLKTEDTAVYYCFRYGIYPDHWGQGTLVTVSS

SEQ ID NO: 72 - protein, 107 aa, synthetic construct
  REGION 1..107: note = h1779_VL.1
DIQMTQSPSSLSASVGDRVTITCLASEGISNDVAWYQQKPGKAPKLLIYAASRLQDGVPSRFSGSGSGTDFTFTISSLQPEDIATYYCQQGYKTPLTFGQGTKLEIK

SEQ ID NO: 73 - protein, 107 aa, synthetic construct
  REGION 1..107: note = h1779_VL.1A
DIQMTQSPSSLSASVGDRVTITCLASEGISNDVAWYQQKPGKSPKLLIYAASRLQDGVPSRFSGSGSGTDFTFTISSLQPEDIATYYCQQGYKTPLTFGQGTKLEIK

SEQ ID NO: 74 - protein, 107 aa, synthetic construct
  REGION 1..107: note = h1779_VL.1B
DIQMTQSPSSLSASVGDRVTITCLASEGISNDVAWYQQKPGKSPKLLVYAASRLQDGVPSRFSGSGSGTDYTFTISSLQPEDIATYYCQQGYKTPLTFGQGTKLEIK

SEQ ID NO: 75 - protein, 120 aa, synthetic construct
  REGION 1..120: note = h1779_VH.1
EVQLVQSGAEVKKPGASVKVSCKASGYTFTDYIIHWVRQAPGQGLEWMGYFNPNSGGSNYNENFKRRVTMTRDTSISTAYMELSRLRSEDTAVYYCARRIAWDHWYFDFWGQGTMVTVSS

SEQ ID NO: 76 - protein, 120 aa, synthetic construct
  REGION 1..120: note = h1779_VH.1A
EVQLVQSGAEVKKPGASVKVSCKASGYTFTDYIIHWVRQAPGQGLEWMGYFNPNSGGSNYNENFKRRVTMTADKSISTAYMELSRLRSEDTAVYYCARRIAWDHWYFDFWGQGTMVTVSS

SEQ ID NO: 77 - protein, 120 aa, synthetic construct
  REGION 1..120: note = h1779_VH.1B
EVQLVQSGAEVKKPGASVKVSCKASGYTFTDYIIHWVRQAPGQGLEWIGYFNPNSGGSNYNENFKRRATMTADKSISTAYMELSRLRSEDTAVYYCARRIAWDHWYFDFWGQGTMVTVSS

SEQ ID NO: 78 - protein, 120 aa, synthetic construct
  REGION 1..120: note = h1779_VH.1C
EVQLVQSGAEVKKPGASVKVSCKASGYTFTDYIIHWVRQAPGQGLEWIGYFNPNSGGSNYNENFKRRATMTADKSISTAYLEFSRLRSEDTAVYYCARRIAWDHWYFDFWGQGTMVTVSS

SEQ ID NO: 79 - protein, 120 aa, synthetic construct
  REGION 1..120: note = h1779_VH.1D
EVQLVQSGAEVKKPGASVKVSCKASGYTFTDYIIHWVKQAPGQGLEWIGYFNPNSGGSNYNENFKRKATMTADKSISTAYLEFSRLRSEDTAVYYCARRIAWDHWYFDFWGQGTMVTVSS

SEQ ID NO: 80 - protein, 443 aa, synthetic construct
  REGION 1..443: note = Heavy chain sequence of Hu39D10
EVQLVESGGGLVQPGGSLRLSCAVSGLSLTSNSVNWIRQAPGKGLEWVGLIWSNGDTDYNSAIKSRFTISRDTSKSTVYLQMNSLRAEDTAVYYCAREYYGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 214 aa, synthetic construct
  REGION 1..214: note = Light chain sequence of Hu39D10
DIQMTQSPSSLSASVGDRVTITCLASEGISSYLAWYQQKPGKAPKLLIYGANSLQTGVPSRFSGSGSATDYTLTISSLQPEDFATYYCQQSYKFPNTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 17 aa, synthetic construct
  REGION 1..17: note = h1773 antibody body heavy chain HCDR2 variant
RIDPAVGDTKHGPKFQG

(19) United States
(12) Patent Application Publication
(10) Pub. No.: US 2025/0333494 A1
(43) Pub. Date: Oct. 30, 2025

(54) IL-5 ANTIBODY, ANTIGEN BINDING FRAGMENT THEREOF, AND MEDICAL APPLICATION THEREFOR

(71) Applicants: Jiangsu Hengrui Medicine Co., Ltd., Jiangsu (CN); Shanghai Hengrui Pharmaceutical Co., Ltd., Shanghai (CN)

(72) Inventors: Hua Ying, Shanghai (CN); Jinping Shi, Shanghai (CN); Yifang Wang, Shanghai (CN); Qiyue Hu, Shanghai (CN); Hu Ge, Shanghai (CN); Weikang Tao, Shanghai (CN)

(21) Appl. No.: 19/043,179

(22) Filed: Jan. 31, 2025

Related U.S. Application Data
(62) Division of application No. 17/747,627, filed on May 18, 2022, now Pat. No. 12,258,394.
Application No. 17/747,627 is a division of application No. 16/651,639, filed on Mar. 27, 2020, now Pat. No. 11,365,247.

(30) Foreign Application Priority Data
Sep. 29, 2017 (CN) 201710906068.X

(51) Int. Cl. C07K 16/24; A61K 39/00; A61P 11/06

(52) U.S. Cl. CPC C07K 16/244; A61P 11/06; A61K 2039/505; C07K 2317/24; C07K 2317/54; C07K 2317/55; C07K 2317/565; C07K 2317/569; C07K 2317/92

(57) ABSTRACT

Provided are an IL-5 antibody, an antigen binding fragment thereof, and a medical application therefor. The present invention comprises a mouse-derived antibody containing an IL-5 antibody CDR region, a chimeric antibody, a humanized antibody, and a pharmaceutical composition comprising said IL-5 antibody and said antigen binding fragment thereof, as well as the use of the pharmaceutical composition as a drug.

### CROSS-REFERENCE TO RELATED APPLICATIONS

This application is a divisional application of, and claims priority to, U.S. patent application Ser. No. 17/747,627, which was filed on May 18, 2022, and which is a divisional of U.S. patent application Ser. No. 16/651,639, which was filed on Mar. 27, 2020 and is now U.S. Pat. No. 11,365,247, and which is a U.S. National Phase Entry of International PCT Application No. PCT/CN2018/108240, which was filed on Sep. 28, 2018, and which claims the benefit of Chinese Patent Application Serial No. 201710906068.X, which was filed on Sep. 29, 2017. The contents of each of those applications are incorporated herein by reference in their entireties.

### STATEMENT REGARDING SEQUENCE LISTING

This application incorporates by reference the material in the ST.26 XML file titled CHENG-77_Sequence-Listing, which was created on Feb. 1, 2025 and is 107 KB.

### FIELD OF THE INVENTION

The present disclosure relates to IL-5 antibodies and antigen-binding fragments thereof. Further, the present disclosure also relates to chimeric antibodies, humanized antibodies comprising the CDR regions of the IL-5 antibodies, and the present disclosure also relates to a pharmaceutical composition comprising the IL-5 antibody and antigen-binding fragment thereof, and its use as a diagnostic and therapeutic agent for IL-5-related diseases.

### BACKGROUND OF THE INVENTION

Interleukin-5 (IL-5) is one of the important members of the interleukin family, also known as T cell replacing factor (TRF), B cell growth factor-II (BCGF-II), IgA-enhancing factor (IgA-EF), or eosinophil differentiation factor (EDF). It is a homodimeric glycoprotein secreted mainly by helper T cell 2 (Th2). Human IL-5 consists of 134 amino acid residues, including a signal peptide consisting of 22 amino acids and two glycosylation sites. Human IL-5 has 70% identity with murine IL-5 on amino acid level. An active IL-5 is in the form of oligodimer, with two peptide chains linked to each other via disulfide bond(s) and in an antiparallel configuration, while the monomers of IL-5 are not biologically active (Adv Immunol. 1994; 57:145-90).

Eosinophil (EOS) is associated with a variety of inflammatory diseases in lung, including allergic diseases associated with anaphylactic reaction. Among these diseases, asthma is a chronic respiratory inflammatory disease, affecting approximately 300 million patients worldwide, with a morbidity of 10%. Its pathogenesis is associated with a variety of cytokines, and IL-5 and its receptor IL-5R play an important role in the pathogenesis of asthma. There is a large amount of inflammatory cells infiltrating in the bronchopulmonary tissue of patients with asthma, among which eosinophils are most significantly increased. Many studies have shown that eosinophil is one of the major cells leading to airway inflammation in asthma (Curr Opin Pulm Med. 2005 January; 11(1): 1-6). IL-5 plays an extremely important role in the differentiation, maturation, adhesion, infiltration and apoptosis of EOS. A large number of animal studies and clinical studies have shown that IL-5 can activate EOS progenitor cells in the bone marrow and initiate the aggregation of EOS in peripheral blood and airway, leading to chronic inflammation and hyperresponsiveness of the airway (J Immunol. 2014 Oct. 15; 193(8):4043-52). In addition, IL-5 can prolong the survival duration of EOS, enhance its degranulation response to specific stimulating factors (such as IgA or IgG), and mediate the chemotactic activity of eosinophils (J Asthma Allergy. 2015 Nov. 3; 8:125-34). Increased expression of IL-5 was detected in both asthma patients and human bronchial antigen-induced models (Greenfeder et al, Respiratory Research, 2:71-79, 2001). The recombinant human IL-5 protein taken by asthma patients will result in the increased number of eosinophils, the bronchial hyperresponsiveness, and release of toxic particles by eosinophils, indicating that IL-5 is a key factor in the pathogenesis of asthma.

Currently, the most effective method for the treatment of asthma is to inhibit the expression of some key mediators (including IL-5) in asthma via nasal or oral administration of sterols to alleviate inflammation in lung. However, long-term use of sterols has many side effects. It is therefore necessary to find new pharmaceutical targets for the treatment of asthma. Studies have shown that by inhibiting the binding of IL-5 to its receptor, IL-5 antibodies can significantly reduce the accumulation of eosinophils in lung, reduce the level of eosinophils in blood, tissue and sputum, decrease the eosinophil-mediated inflammatory response, improve lung function, and exhibit good efficacy for the treatment of severe eosinophil asthma and recurrent asthma (Drugs. 2017 May; 77(7): 777-784). Currently, only IL-5 antibodies mepolizumab from GSK and reslizumab from Teva Pharma are commercially available. Other antibodies against the IL-5 target are in preclinical research phase. The related patents are for example, WO2017033121, WO2016040007, WO2015095539, WO2012083370, WO2012158954, WO2006046689, WO9621000, WO9535375, etc., However, there is still a need for the improvement in IL-5-induced elimination of eosinophils and the improvement of lung function. Therefore, it is necessary to continue to develop antibodies with high selectivity, high affinity and good efficacy to provide more and preferred anti-IL-5 treatment regimens for asthma.

### SUMMARY OF THE INVENTION

The present disclosure provides monoclonal antibodies or antigen-binding fragments (also referred to as IL-5 binding molecules) that specifically bind to the amino acid sequence of IL-5 or three-dimensional structure.

In one aspect, the disclosure provides a monoclonal antibody or antigen-binding fragment thereof binding to human IL-5, wherein the monoclonal antibody comprises a heavy chain variable region and a light chain variable region, wherein,
(i) the heavy chain variable region comprises HCDR1, HCDR2 and HCDR3 region as set forth in amino acid sequences of SEQ ID NOs: 16-18, or HCDR variants having 3, 2, or 1 amino acid difference(s) from HCDR1, HCDR2 and HCDR3 region as set forth in SEQ ID NOs: 16-18, respectively; and the light chain variable region comprises LCDR1, LCDR2 and LCDR3 region as set forth in amino acid sequences of SEQ ID NOs: 19-21, or LCDR variants having 3, 2, or 1 amino acid difference(s) from LCDR1, LCDR2 and LCDR3 region as set forth in SEQ ID NOs: 19-21, respectively; or
(ii) the heavy chain variable region comprises HCDR1, HCDR2 and HCDR3 region as set forth in amino acid sequences of SEQ ID NOs: 22-24, or HCDR variants having 3, 2, or 1 amino acid difference(s) from HCDR1, HCDR2 and HCDR3 region as set forth in SEQ ID NOs: 22-24, respectively; and the light chain variable region comprises LCDR1, LCDR2 and LCDR3 region as set forth in amino acid sequences of SEQ ID NOs: 25-27, or LCDR variants having 3, 2, or 1 amino acid difference(s) from LCDR1, LCDR2 and LCDR3 region as set forth in SEQ ID NOs: 25-27, respectively; or
(iii) the heavy chain variable region comprises HCDR1, HCDR2 and HCDR3 region as set forth in amino acid sequences of SEQ ID NOs: 28-30, or HCDR variants having 3, 2, or 1 amino acid difference(s) from HCDR1, HCDR2 and HCDR3 region as set forth in SEQ ID NOs: 28-30, respectively; and the light chain variable region comprises LCDR1, LCDR2 and LCDR3 region as set forth in amino acid sequences of SEQ ID NOs: 31-33, or LCDR variants having 3, 2, or 1 amino acid difference(s) from LCDR1, LCDR2 and LCDR3 region as set forth in SEQ ID NOs: 31-33, respectively; or
(iv) the heavy chain variable region comprises HCDR1, HCDR2 and HCDR3 region as set forth in amino acid sequences of SEQ ID NOs: 34-36, or HCDR variants having 3, 2, or 1 amino acid difference(s) from HCDR1, HCDR2 and HCDR3 region as set forth in SEQ ID NOs: 34-36, respectively; and the light chain variable region comprises LCDR1, LCDR2 and LCDR3 region as set forth in amino acid sequences of SEQ ID NOs: 37-39, or LCDR variants having 3, 2, or 1 amino acid difference(s) from LCDR1, LCDR2 and LCDR3 region as set forth in SEQ ID NOs: 37-39, respectively; or
(v) the heavy chain variable region comprises HCDR1, HCDR2 and HCDR3 region as set forth in amino acid sequences of SEQ ID NOs: 40-42, or HCDR variants having 3, 2, or 1 amino acid difference(s) from HCDR1, HCDR2 and HCDR3 region as set forth in SEQ ID NOs: 40-42, respectively; and the light chain variable region comprises LCDR1, LCDR2 and LCDR3 region as set forth in amino acid sequences of SEQ ID NOs: 43-45, or LCDR variants having 3, 2, or 1 amino acid difference(s) from LCDR1, LCDR2 and LCDR3 region as set forth in SEQ ID NOs: 43-45, respectively; or
(vi) the heavy chain variable region comprises HCDR1, HCDR2 and HCDR3 region as set forth in amino acid sequences of SEQ ID NOs: 34-36, or HCDR variants having 3, 2, or 1 amino acid difference(s) from HCDR1, HCDR2 and HCDR3 region as set forth in SEQ ID NOs: 34, 82 and 36, respectively; and the light chain variable region comprises LCDR1, LCDR2 and LCDR3 region as set forth in amino acid sequences of SEQ ID NOs: 37-39, or LCDR variants having 3, 2, or 1 amino acid difference(s) from LCDR1, LCDR2 and LCDR3 region as set forth in SEQ ID NOs: 37-39, respectively.

In some embodiments, the variants of the monoclonal antibody or antigen-binding fragment CDRs (including 3 heavy chain CDRs and 3 light chain CDRs) having 3, 2 or 1 amino acid difference(s) are those that are obtained by affinity maturation methods.

In some embodiments, the monoclonal antibodies or antigen-binding fragments bind to IL-5 with an affinity (KD) of less than 10⁻⁸ M, less than 10⁻⁹ M, less than 10⁻¹⁰ M, or less than 10⁻¹¹ M.

In some embodiments, the monoclonal antibody or antigen-binding fragment specifically binds to human IL-5, the monoclonal antibody comprises a heavy chain variable region and a light chain variable region, wherein:
(vii) the heavy chain variable region comprises HCDR1, HCDR2 and HCDR3 region as set forth in SEQ ID NOs: 16-18, and the light chain variable region comprises LCDRI, LCDR2 and LCDR3 region as set forth in SEQ ID NOs: 19-21; or
(viii) the heavy chain variable region comprises HCDR1, HCDR2 and HCDR3 region as set forth in SEQ ID NOs: 22-24, and the light chain variable region comprises LCDR1, LCDR2 and LCDR3 region as set forth in SEQ ID NOs: 25-27; or
(ix) the heavy chain variable region comprises HCDR1, HCDR2 and HCDR3 region as set forth in SEQ ID NOs: 28-30, and the light chain variable region comprises LCDR1, LCDR2 and LCDR3 region as set forth in SEQ ID NOs: 31-33; or
(x) the heavy chain variable region comprises HCDR1, HCDR2 and HCDR3 region as set forth in SEQ ID NOs: 34-36, and the light chain variable region comprises LCDRI, LCDR2 and LCDR3 region as set forth in SEQ ID NOs: 37-39; or
(xi) the heavy chain variable region comprises HCDR1, HCDR2 and HCDR3 region as set forth in SEQ ID NOs: 40-42, and the light chain variable region comprises LCDR1, LCDR2 and LCDR3 region as set forth in SEQ ID NOs: 43-45; or
(xii) the heavy chain variable region comprises HCDR1, HCDR2 and HCDR3 region as set forth in SEQ ID NOs: 34, 82 and 36, and the light chain variable region comprises LCDR1, LCDR2 and LCDR3 region as set forth in SEQ ID NOs: 37-39.

In some embodiments, the monoclonal antibody is recombinant antibody.

In some embodiments, the monoclonal antibody is selected from the group consisting of murine antibody, chimeric antibody, recombinant antibody of a humanized antibody, or antigen-binding fragment thereof.

In some embodiments, the light and heavy chain FR region sequences on the humanized antibody light and heavy chain variable region are respectively derived from human germline light and heavy chain, or mutated sequences thereof.

In some embodiments of the monoclonal antibody or antigen-binding fragment thereof, the humanized antibody comprises a heavy chain variable region of SEQ ID NO: 49, 57, 63, 69 or 75, or a variant thereof; the variant has a 1-10 amino acid mutation(s) on the heavy chain variable region as set forth in SEQ ID NO: 49, 57, 63, 69 or 75.

In some embodiments of the monoclonal antibody or antigen-binding fragment thereof, the variant has 1-10 amino acid back mutations on the FR region of the heavy chain variable region as set forth in SEQ ID NO: 49, 57, 63, 69 or 75; preferably, the back mutation is selected from the group consisting of S49T, V93T and K98S, or a combination thereof on the heavy chain variable region of SEQ ID NO: 49, or the back mutation is selected from the group consisting of S49T, V93T and K98T, or a combination thereof on the heavy chain variable region of SEQ ID NO: 57, or the back mutation is selected from the group consisting of R38K, M48I, R67K, V68A, M70L, R72V, T74K and L83F, or a combination thereof on the heavy chain variable region of SEQ ID NO:63, or the back mutation is selected from the group consisting of F29I, R38K, V481, R72A, T97F and N55V, or a combination thereof on the heavy chain variable region of SEQ ID NO:69, or the back mutation is selected from the group consisting of R38K, M48I, R67K, V68A, R72A, T74K, M81L, L83F and D89E, or a combination thereof on the heavy chain variable region of SEQ ID NO:75.

In some embodiments of the monoclonal antibody or antigen-binding fragment thereof, the humanized antibody comprises a heavy chain variable region of SEQ ID NO:50 or 51, or comprises a heavy chain variable region of SEQ ID NO: 58 or 59, or comprises a heavy chain variable region selected from any one of SEQ ID NO: 64, 65 and 66, or comprises a heavy chain variable region of SEQ ID NO:70 or 71, or comprises a heavy chain variable region selected from any one of SEQ ID NOs: 76 to 79.

In some embodiments of the monoclonal antibody or antigen-binding fragment thereof, the humanized antibody comprises a light chain variable region of SEQ ID NO: 46, 54, 60, 67 or 72 or variant thereof; the variant has 1-10 amino acid change(s) on the light chain variable region as set forth in SEQ ID NO: 46, 54, 60, 67 or 72.

In some embodiments of the monoclonal antibody or antigen-binding fragment thereof, the variant has 1-10 amino acid back mutation(s) on the FR region of the light chain variable region as set forth in SEQ ID NO: 46, 54, 60, 67 or 72; preferably, the back mutation is selected from the group consisting of A43S, L47V, G66R, T69S, F71Y and Y87F or a combination thereof on the light chain variable region of SEQ ID NO:46; or the back mutation is selected from the group consisting of A43S, L47M, F71Y and Y87F or a combination thereof on the light chain variable region of SEQ ID NO: 54; or the back mutation is selected from the group consisting of EID, I2T, 157V, V84T and Y86F or a combination thereof on the light chain variable region of SEQ ID NO: 60; or the back mutation is selected from the group consisting of M4L, A42S, L45P and L46W or a combination thereof on the light chain variable region of SEQ ID NO: 67; or the back mutation is selected from the group consisting of A43S, 148V and F71Y or a combination thereof on the light chain variable region of SEQ ID NO: 72.

In some embodiments of the monoclonal antibody or antigen-binding fragment thereof, the humanized antibody comprises a light chain variable region of SEQ ID NO: 47 or 48; or comprises a light chain variable region of SEQ ID NO: 55 or 56; or comprises a light chain variable region of SEQ ID NO: 61 or 62; or comprises a light chain variable region of SEQ ID NO: 68; or comprises a light chain variable region of SEQ ID NO: 73 or 74.

In some embodiments of the monoclonal antibody or antigen-binding fragment thereof, the humanized antibody comprises:
a heavy chain variable region selected from any one of SEQ ID NOs: 49-51 and a light chain variable region selected from any one of SEQ ID NOs: 46-48; or
a heavy chain variable region selected from any one of SEQ ID NOs: 57-59 and a light chain variable region selected from any one of SEQ ID NOs: 54-56; or
a heavy chain variable region selected from any one of SEQ ID NOs: 63-66 and a light chain variable region selected from any one of SEQ ID NOs: 60-62; or
a heavy chain variable region selected from any one of SEQ ID NOs: 69-71 and a light chain variable region selected from any one of SEQ ID NOs: 67-68; or
a heavy chain variable region selected from any one of SEQ ID NOs: 75-79 and a light chain variable region selected from any one of SEQ ID NOs: 72-74.

In some embodiments of the monoclonal antibody or antigen-binding fragment thereof, the antibody is a full-length antibody, further comprises a human antibody constant region, wherein the heavy chain constant region is preferably human IgG1, IgG2, IgG3, and IgG4 antibody heavy constant region. More preferably, the full-length antibody comprises a human antibody heavy chain constant region as set forth in SEQ ID NO: 52 and a human light chain constant region as set forth in SEQ ID NO:53.

In some embodiments, the antigen-binding fragment is selected from the group consisting of Fab, Fab′, F(ab′) 2, single-chain antibody (scFv), dimerized V region (diabody), disulfide-stabilized V region (dsFv) and a peptide comprising CDRs.

The present disclosure also provides an isolated monoclonal antibody or antigen-binding fragment thereof, which competes for binding to human IL-5 with the monoclonal antibody or antigen-binding fragment thereof described above.

The present disclosure also provides a pharmaceutical composition comprising a therapeutically effective amount of the monoclonal antibody or antigen-binding fragment thereof according to the present disclosure, and one or more pharmaceutically acceptable carriers, diluents, buffers or excipients. The amount of the monoclonal antibody or antigen-binding fragment thereof contained in the unit dose of the pharmaceutical composition is preferably from 0.1 to 2000 mg, more preferably from 1 to 1000 mg.

The present disclosure also provides an isolated nucleic acid molecule encoding the monoclonal antibody or antigen-binding fragment thereof according to the present disclosure.

The present disclosure also provides a recombinant vector comprising the nucleic acid molecule described above.

The present disclosure also provides a host cell transformed with the recombinant vector according to the present disclosure, the host cell being selected from the group consisting of prokaryotic cells and eukaryotic cells, preferably eukaryotic cells, more preferably mammalian cells.

The present disclosure also provides a method for producing the monoclonal antibody or antigen-binding fragment thereof according to the present disclosure, the method comprises cultivating the above host cell in a culture to form and accumulate the above monoclonal antibody or antigen-binding fragment thereof, and recovering the monoclonal antibody or antigen-binding fragment thereof from the culture.

The present disclosure also provides a method for detecting or determining human IL-5, the method comprises using the above monoclonal antibody or antigen-binding fragments thereof.

The present disclosure also provides an agent for detecting or determining human IL-5, which comprises the monoclonal antibody or antigen-binding fragment thereof according to any one of the above.

The present disclosure also provides a diagnostic agent for a disease associated with human IL-5, the diagnostic agent comprises the above monoclonal antibody or antigen-binding fragment thereof.

The present disclosure also provides a method for diagnosing a disease associated with human IL-5, the method comprises detecting or determining human IL-5 or IL-5 positive cells using the above monoclonal antibody or antigen-binding fragment thereof.

The present disclosure also provides use of the above monoclonal antibody or antigen-binding fragment thereof for the preparation of a diagnostic agent for a disease associated with human IL-5.

The present disclosure also provides a medicament for treating a disease associated with human IL-5, comprising the above monoclonal antibody or antigen-binding fragment thereof, or comprising the above pharmaceutical composition, or comprising the above nucleic acid molecule.

The present disclosure also provides a method of treating a disease associated with human IL-5, the method comprises administering to a subject a pharmaceutically effective amount of the above monoclonal antibody or antigen-binding fragment thereof, or a pharmaceutical composition comprising the same, or the above nucleic acid molecule to prevent or treat the disease associated with human IL-5.

The present disclosure also provides use of the above monoclonal antibody or antigen-binding fragment thereof, or the pharmaceutical composition comprising the same, or the above nucleic acid molecule for preparing a therapeutic agent for a disease associated with human IL-5.

The above disease or condition is preferably selected from the group consisting of asthma, malignant attack of asthma, chronic pneumonia, allergic rhinitis, allergic bronchopulmonary aspergillosis, eosinophilia, Churg-Strauss syndrome, atopic dermatitis, onchocerciasis dermatitis, intermittent angioedema, eosinophilic myalgia syndrome, eosinophilic gastroenteritis, helminth infection, Hodgkin's disease, nasal polyps, Loeffler's syndrome, urticaria, eosinophil hyperplastic bronchitis, nodular arteritis, sinusitis, eosinophilic esophagitis, allergic eosinophilic esophagitis, allergic conjunctivitis, onchocerciasis dermatitis, endometriosis and steroid dependent eosinophilic bronchitis.

The IL-5 monoclonal antibodies or antigen-binding fragments of the present disclosure have high specificity and high affinity with IL-5. The humanized antibodies have greatly reduced immunogenicity and completely retain specificity from the murine antibody and exhibit high affinity and excellent activities in vitro and in vivo.

The IL-5 monoclonal antibodies or antigen-binding fragments of the present disclosure have good selectivity for merely specifically recognizing IL5.

The IL-5 monoclonal antibodies or antigen-binding fragments of the present disclosure have good metabolic dynamic characteristics in rats, exhibit long half-life, and high bioavailability.

The IL-5 humanized antibody molecules of the present disclosure have good long-term stability, no obvious abnormal chemical modification, no obvious aggregation at high concentration, and high purity and thermal stability.

In addition to reducing the proliferation of eosinophils, the IL-5 monoclonal antibodies or antigen-binding fragments of the present disclosure have good properties in improving lung function.

### DESCRIPTION OF THE DRAWINGS

FIG. 1: IL-5 antibodies block the binding of IL-5 to IL-5 receptor in FACS experiment;

FIG. 2: Detection of binding specificity of IL-5 antibodies to Th2 cytokine;

FIG. 3: IL-5 antibodies enhance respiratory intermittent value (Penh). G1: normal control group (PBS); G2: model group (IgG); G3: h1705-008 antibody 10mpk group; G4: h1705-008 antibody 2mpk group; G5: h1706-009 antibody 10mpk group; G6: h1706-009 antibody 2mpk group; G7: Hu39D10 10mpk group; *p<0.05, **<0.01 (compared with G2 group by ANOVA/Bonferroni);

FIG. 4A: The level of BALF eosinophils in lung of asthma mice; FIG. 4B: Scores of thickness of asthma mice tracheal mucosa. G1: normal control group; G2: model group; G3: h1705-008 antibody 10mpk group; G4: h1705-008 antibody 2mpk group; G5: h1706-009 antibody 10mpk group; G6: h1706-009 antibody 2mpk group; G7: Hu39D10 10mpk group; FIG. 4C: Percentage of BALF eosinophils in lung of asthma mice;

FIG. 5A and FIG. 5B show the ability of IL5 mAb to reduce the level of eosinophils in BALF.

### DETAILED DESCRIPTION OF THE INVENTION

### 1. Terminology

In order to more easily understand the present disclosure, certain technical and scientific terms are specifically defined below. Unless otherwise defined explicitly herein, all other technical and scientific terms used herein have the meaning commonly understood by one of ordinary skill in the art to which this disclosure belongs.

Three-letter codes and one-letter codes for amino acids used in the present disclosure are as described in J. biol. chem, 243, p 3558 (1968).

As used herein, “antibody” refers to immunoglobulin, a four-peptide chain structure connected together by disulfide bond between two identical heavy chains and two identical light chains. Different immunoglobulin heavy chain constant regions exhibit different amino acid compositions and rank orders, hence present different antigenicity. Accordingly, immunoglobulins can be divided into five types, or called immunoglobulin isotypes, namely IgM, IgD, IgG, IgA and IgE, with heavy chain μ, δ, γ, α and ε, respectively. According to its amino acid composition of hinge region and the number and location of heavy chain disulfide bonds, the same type of Ig can further be divided into different sub-types, for example, IgG can be divided into IgG1, IgG2, IgG3 and IgG4. Light chain can be divided into κ or λ chain, based on different constant region. Each of five types of Ig may has κ or λ chain.

In the present disclosure, the antibody light chain mentioned in present disclosure further comprises a light chain constant region, which comprises human or murine κ, λ chain or a variant thereof.

In the present disclosure, the antibody heavy chain mentioned in present disclosure further comprises a heavy chain constant region, which comprises human or murine IgG1, IgG 2, IgG 3, IgG 4 or a variant thereof.

About 110 amino acid sequences adjacent to the N-terminus of the antibody heavy and light chains are highly variable, known as variable region (Fv region); the rest of amino acid sequences close to the C-terminus are relatively stable, known as constant region. The variable region includes three hypervariable regions (HVRs) and four relatively conservative framework regions (FRs). The three hypervariable regions which determine the specificity of the antibody are also known as the complementarity determining regions (CDRs). Each light chain variable region (LCVR) and each heavy chain variable region (HCVR) consist of three CDR regions and four FR regions, with sequential order from the amino terminus to carboxyl terminus in the following order: FR1, CDR1, FR2, CDR2, FR3, CDR3, and FR4. The three CDR regions of the light chain refer to LCDR1, LCDR2, and LCDR3, and the three CDR regions of the heavy chain refer to HCDR1, HCDR2, and HCDR3. The number and position of CDR amino acid residues in the LCVR and HCVR regions of the antibody or antigen binding fragments herein comply with known Kabat numbering criteria (LCDR1-3, HCDR1-3).

The antibody of the present disclosure comprises murine antibody, chimeric antibody and humanized antibody, preferably is humanized antibody.

The term “murine antibody” in the present disclosure refers to anti-human IL-5 monoclonal antibody prepared according to the knowledge and skills of the field. During the preparation, test subject may be injected with IL-5 antigen, and then a hybridoma expressing the antibody which possesses desired sequence or functional characteristics is isolated. In a preferred embodiment of the present disclosure, the murine IL-5 antibody or antigen binding fragment thereof further comprises light chain constant region of murine κ, λ chain or a variant thereof, or further comprises heavy chain constant region of murine IgG1, IgG2, IgG3 or IgG4, or a variant thereof.

The term “chimeric antibody”, is an antibody by fusing the variable region of murine antibody with the constant region of human antibody, and the chimeric antibody can alleviate the murine antibody-induced immune response. To establish a chimeric antibody, a hybridoma secreting specific murine monoclonal antibody may be established and a variable region gene is cloned from the murine hybridoma. Then a desired constant region gene of human antibody can be cloned, and connected with a variable region gene of murine to form a chimeric gene which can be subsequently inserted into an expression vector. Finally the chimeric antibody molecule will be expressed in the eukaryotic or prokaryotic system. In a preferred embodiment of the present disclosure, the light chain of the IL-5 chimeric antibody further comprises a light chain constant region derived from human κ, λ chain or a variant thereof. The heavy chain of IL-5 chimeric antibody further comprises a heave chain constant region derived from human IgG1, IgG2, IgG3, IgG4 or a variant thereof, preferably comprises a heavy chain constant region derived from human IgG1, IgG2 or IgG4, or comprises a heavy chain constant region variant of human IgG1, IgG2 or IgG4 with amino acid mutation(s), such as YTE mutation(s) or back-mutation(s).

The term “humanized antibody”, refers to an antibody generated by grafting murine CDR sequences into human antibody variable region framework, i.e., an antibody produced in different types of human germline antibody framework sequences. Humanized antibody can overcome heterologous responses induced by large number of murine protein components carried by chimeric antibody. Such framework sequences can be obtained from public DNA database covering germline antibody gene sequences or published references. For example, germline DNA sequences of human heavy and light chain variable region genes can be found in “VBase” human germline sequence database (available on web www.mrccpe.com.ac.uk/vbase), as well as in Kabat, E A, et al. 1991 Sequences of Proteins of Immunological Interest, 5th Ed. To avoid a decrease in activity caused by the decreased immunogenicity, the framework sequences in the variable region of human antibody may be subjected to minimal reverse mutations or back mutations to maintain the activity. The humanized antibody of the present disclosure also comprises humanized antibody on which CDR affinity maturation is performed by phage display. In a preferred embodiment of the present disclosure, the CDR sequence of the IL-5 humanized antibody is selected from the group consisting of SEQ ID NOs: 16-21, 22-27, 28-33, 34-39 and 40-45. The human antibody variable region framework is designed and selected, wherein the FR region sequence on the antibody heavy chain variable region is derived from the human germline heavy chain sequence and the human germline light chain sequence. To avoid a decrease in activity caused by the decreased immunogenicity, the human antibody variable region may be subjected to minimal reverse mutations (back mutations, that is, the FR region amino acid residues derived from human antibody are replaced with amino acid residues corresponding to the original antibody) to maintain the activity.

The graft of CDR can result in the decrease of the affinity of the resulting IL-5 antibody or antigen binding fragment thereof to the antigen due to the change in the framework residues contacted with the antigen. Such interactions may be the result of highly somatic mutations. Therefore, it may still be necessary to transfer the donor framework amino acids to the humanized antibody framework. The amino acid residues derived from non-human IL-5 antibody or antigen binding fragment thereof, that are involved in antigen binding, can be identified by checking the sequence and structure of murine monoclonal antibody variable region. The amino acid residues in donor CDR framework that are different from those in the germ lines can be considered to be related. If it is not possible to determine the most closely related germ line, the sequence can be compared with the common sequence shared among the subtypes or with the common sequence of murine sequences having high similarity percentage. Rare framework residues are thought to be the result of a high mutation in somatic cells, which play an important role in binding.

As used herein, “antigen-binding fragment” or “functional fragment” refers to one or more fragment(s) of antibody retaining the binding ability to the antigen (e.g. IL-5). It has been shown that fragments of full-length antibody can be used to achieve function of binding with an antigen. The examples of binding fragments in the term “antigen binding fragment” include (i) Fab fragment, a monovalent fragment composed of VL, VH, CL and CHI domain; (ii) F(ab′)₂ fragment, a bivalent fragment comprising two Fab fragments linked by a disulphide bond in hinge region; (iii) Fd fragment, consisting of VH and CHI domains; (iv) Fv fragment, consisting of VH and VL domains of one-arm antibody; (v) single domain or dAb fragment (Ward et al. (1989) Nature341: 544-546) composed of VH domain; and (vi) a separate complementary determining region (CDR) and (vii) a combination of two or more separate CDRs optionally linked by a synthetic linker. In addition, although the VL domain and VH domain of the Fv fragment are encoded by two separate genes, they can be linked by a synthetic linker by using recombinant methods, thereby generating a single protein chain of a monovalent molecular formed by pairing the VL and VH domain (referred to as single chain Fv (scFv); see, e.g., Bird et al. (1988); Science 242:423-426 and Huston et al (1988) Proc. Natl. Acad. Sci USA85: 5879-5883). This single chain antibody is also intended to be included in the term “antigen binding fragment” of the antibody. Such antibody fragments are obtained using conventional techniques known in the field, and screened for functional fragments by using the same method as that for an intact antibody. Antigen binding portions can be produced by recombinant DNA technology or by enzymatic or chemical disruption of an intact immunoglobulin. Antibodies can be in the form of different isotypes, e.g., IgG (e.g., IgG1, IgG2, IgG3 or lgG4 subtype), IgA1, IgA2, IgD, IgE or IgM antibody.

The antigen-binding fragment in the present disclosure includes Fab, F(ab′)2, Fab′, single-chain antibody (scFv), dimerized V region (diabody), disulfide stabilized V region (dsFv) and CDR-containing peptide.

Fab is an antibody fragment obtained by treating an IgG antibody molecule with a papain (which cleaves the amino acid residue at position 224 of the H chain). The Fab fragment has a molecular weight of about 50,000 and has antigen binding activity, in which about a half of the N-terminal side of H chain and the entire L chain are bound together through a disulfide bond.

The Fab of the present disclosure can be produced by treating the monoclonal antibody of the present invention which specifically recognizes human IL-5 and binds to the amino acid sequence of extracellular region or three-dimensional structure thereof with papain. Also, the Fab can be produced by inserting DNA encoding Fab of the antibody into a prokaryotic expression vector or eukaryotic expression vector and introducing the vector into a prokaryote or eukaryote to express the Fab.

F(ab′)2 is an antibody fragment having a molecular weight of about 100,000 and having antigen binding activity and comprising two Fab regions which are bound at the hinge position, F(ab′)2 is obtained by digesting the downstream part of the two disulfide bonds in the hinge region of IgG with pepsin.

The F(ab′)2 of the present disclosure can be produced by treating the monoclonal antibody of the present disclosure which specifically recognizes human IL-5 and binds to the amino acid sequence of extracellular region or three-dimensional structure thereof with pepsin. Also, the F(ab′)2 can be produced by binding the Fab′ described below via a thioether bond or a disulfide bond.

Fab′ is an antibody fragment having a molecular weight of about 50,000 and having antigen binding activity. Fab′ is obtained by cleaving a disulfide bond at the hinge region of the above F(ab′)2. The Fab′ of the present disclosure can be produced by treating the F(ab′)2 of the present invention which specifically recognizes IL-5 and binds to the amino acid sequence of extracellular region or three-dimensional structure thereof with a reducing agent, such as dithiothreitol.

Also, the Fab′ can be produced by inserting DNA encoding Fab′ fragment of the antibody into a prokaryotic expression vector or eukaryotic expression vector and introducing the vector into a prokaryote or eukaryote to express the Fab′.

The term “single chain antibody”, “single chain Fv” or “scFv” refers to a molecule comprising an antibody heavy chain variable domain (or region; VH) and an antibody light chain variable domain (or region; VL) connected by a linker. Such scFv molecules have the general structure of NH₂-VL-linker-VH-COOH or NH₂-VH-linker-VL-COOH. A suitable linker in the prior art consists of repeated GGGGS amino acid sequence or variant thereof, for example, using a variant with 1-4 repeats (Holliger et al. (1993), Proc. Natl. Acad. Sci. USA 90:6444-6448). Other linkers that can be used for the present disclosure are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol.

The scFv of the present disclosure can be produced by the following steps: obtaining cDNAs encoding VH and VL of the monoclonal antibody of the present disclosure which specifically recognizes human IL-5 and binds to the amino acid sequence of extracellular region or three-dimensional structure thereof, constructing DNA encoding scFv, inserting the DNA into a prokaryotic expression vector or eukaryotic expression vector, and then introducing the expression vector into a prokaryote or eukaryote to express the scFv.

A diabody is an antibody fragment wherein the scFv is dimerized, and is an antibody fragment having divalent antigen binding activity. In the divalent antigen binding activity, two antigens may be the same or different.

The diabody of the present disclosure can be produced by the following steps, obtaining cDNAs encoding VH and VL of the monoclonal antibody of the present disclosure which specifically recognizes human IL-5 and binds to the amino acid sequence of extracellular region or three-dimensional structure thereof, constructing DNA encoding scFv so that the length of the linker peptide is 8 or less amino acid residues, inserting the DNA into a prokaryotic expression vector or eukaryotic expression vector, and then introducing the expression vector into a prokaryote or eukaryote to express the diabody.

A dsFv is obtained by substituting one amino acid residue in each of VH and VL with a cysteine residue, and then connecting the substituted polypeptides via a disulfide bond between the two cysteine residues. The amino acid residue to be substituted with a cysteine residue can be selected based on three-dimensional structure prediction of the antibody in accordance with known methods (Protein Engineering, 7, 697 (1994)).

The dsFv of the present disclosure can be produced by the following steps: obtaining cDNAs encoding VH and VL of the monoclonal antibody of the present disclosure which specifically recognizes human IL-5 and binds to the amino acid sequence of extracellular region or three-dimensional structure thereof, constructing DNA encoding dsFv, inserting the DNA into a prokaryotic expression vector or eukaryotic expression vector, and then introducing the expression vector into a prokaryote or eukaryote to express the dsFv.

A CDR-containing peptide is constructed by one or more region(s) of CDRs of VH and VL. Peptides comprising several CDRs can be joined directly or via a suitable peptide linker.

The CDR-containing peptide of the present disclosure can be produced by the steps of: constructing a DNA encoding the CDRs of VH and VL of the monoclonal antibody of the present disclosure which specifically recognizes human IL-5 and binds to the amino acid sequence of extracellular region amino acid sequence or three-dimensional structure thereof, inserting the DNA into a prokaryotic expression vector or eukaryotic expression vector, and then introducing the expression vector into a prokaryote or eukaryote to express the peptide. The CDR-containing peptide can also be produced by a chemical synthesis method such as Fmoc method or tBoc method.

The term “antibody framework” as used herein refers to part of the variable domain, either VL or VH, which serves as a scaffold for the antigen binding loops (CDRs) of this variable domain. In essence it is the variable domain without the CDRs.

The term “amino acid difference” refers to differences on one or more amino acid position(s) along the length of a polypeptide fragment between a polypeptide and a variant thereof, wherein the variant may be obtained by replacing, inserting or deleting one or more amino acid(s) on the polypeptide.

The term “epitope” or “antigenic determinant” refers to a site on an antigen to which an immunoglobulin or antibody specifically binds (e.g., a specific site on the IL-5 molecule). Epitopes typically include at least 3, 4, 5, 6, 7, 8, 9, 10, 11, 12, 13, 14 or 15 consecutive or non-consecutive amino acids in a unique tertiary conformation. See, for example, Epitope Mapping Protocols in Methods in Molecular Biology, Vol. 66, ed. G. E. Morris (1996).

The term “specifically bind to”, “selectively bind to”, “selectively binds to” or “specifically binds to” refers to the binding of an antibody to a predetermined epitope on an antigen. Typically, the antibody binds with an affinity (KD) of less than about 10⁻⁸ M, for example, less than about 10⁻⁹ M, 10⁻¹⁰ M or 10⁻¹¹ M or even less.

The term “KD” refers to the dissociation equilibrium constant for particular antibody-antigen interaction. Typically, the antibody of the present disclosure binds to IL-5 with a dissociation equilibrium constant (KD) of less than about 10⁻⁷ M, such as less than about 10⁻⁸ M, 10⁻⁹ M or 10⁻¹⁰ M or even less, for example, as determined using surface plasmon resonance (SPR) techniques in a BIACORE instrument.

When the term “competition” is used in the context of antigen binding proteins (e.g., neutralizing antigen binding proteins or neutralizing antibodies) that compete for the same epitope, it means that competition occurs among the antigen binding proteins, which is determined by the following assays: an antigen binding protein to be tested (e.g., an antibody or immunologically functional fragment thereof) prevents or inhibits (e.g., reduces) the specific binding between a reference antigen binding protein (e.g., a ligand or reference antibody) and a common antigen (e.g., an IL-5 antigen or fragment thereof). Numerous types of competitive binding assays are available to determine whether an antigen binding protein competes with another. These assays are, for example, solid phase direct or indirect radioimmunoassay (RIA), solid phase direct or indirect enzyme immunoassay (EIA), Sandwich competition assay (see, e.g., Stahli et al, 1983, Methods in Enzymology 9:242-253); solid phase direct biotin-avidin EIA (see, e.g., Kirkland et al, 1986, J. Immunol. 137:3614-3619), solid phase direct labeling assay, solid phase direct labeling sandwich assay (see, e.g., Harlow and Lane, 1988, Antibodies, A Laboratory Manual, Cold Spring Harbor Press); solid phase direct labeling RIA with I-125 label (see, e.g., Morel et al, 1988, Molec. Immunol. 25:7-15); solid phase direct biotin-avidin EIA (see, e.g., Cheung, et al, 1990, Virology 176:546-552); and direct labeling RIA (Moldenhauer et al, 1990, Scand. J. Immunol. 32:77-82). Typically, the assay involves the use of a purified antigen (either on a solid surface or on a cell surface) capable of binding to both an unlabeled antigen binding protein to be tested and a labeled reference antigen binding protein. Competitive inhibition is determined by measuring the amount of label bound to the solid surface or to the cell in the presence of the antigen binding protein to be tested. Usually, the antigen binding protein to be tested is present in excess. Antigen binding proteins identified by competitive assay (competing with the antigen binding protein) includes: antigen binding proteins that bind to the same epitope as the reference antigen binding protein; and antigen binding proteins that bind to an epitope that is sufficiently close to the epitope to which the reference antigen binding protein binds, where the two epitopes spatially interfere with each other to hinder the binding. Additional details regarding methods for determining competitive binding are provided in the Examples herein. Typically, when a competing antigen binding protein is present in excess, it will inhibit (e.g., reduce) at least 40-45%, 45-50%, 50-55%, 55-60%, 60-65%, 65-70%, 70-75% or 75% or even more of the specific binding between the reference antigen binding protein and the common antigen. In some cases, the binding is inhibited by at least 80-85%, 85-90%, 90-95%, 95-97%, or 97% or even more.

The term “nucleic acid molecule,” as used herein refers to DNA molecules and RNA molecules. A nucleic acid molecule may be single-stranded or double-stranded, but preferably is double-stranded DNA. A nucleic acid is “operably linked” when it is placed into a functional relationship with another nucleic acid sequence. For instance, a promoter or enhancer is operably linked to a coding sequence if it affects the transcription of the sequence.

The term “vector” refers to a nucleic acid molecule capable of transporting another nucleic acid to which it has been linked. In one embodiment, the vector is a “plasmid,” which refers to a circular double stranded DNA loop into which additional DNA segments may be ligated. In another embodiment, the vector is a viral vector, wherein additional DNA segments may be ligated into the viral genome. The vectors disclosed herein are capable of self-replicating in the host cell into which they are introduced (e.g., bacterial vectors having a bacterial replication origin and episomal mammalian vectors), or may be integrated into the genome of a host cell upon introduction into the host cell, and thereby are replicated along with the host genome (e.g., non-episomal mammalian vectors).

Methods for producing and purifying antibodies and antigen-binding fragments are well known in the art and can be found, for example, in Antibodies, A Laboratory Manual, Cold Spring Harbor Laboratory Press, chapters 5-8 and 15. For example, mice can be immunized with human IL-5 or fragments thereof, and the resulting antibodies can then be renatured, purified, and sequenced for amino acid sequences by using conventional methods well known in the art. Antigen-binding fragments can also be prepared by conventional methods. The antibodies or antigen binding fragments of the present disclosure are engineered to contain one or more human FR region(s) on CDRs derived from a non-human antibody. Human FR germline sequences can be obtained by aligning human antibody variable germline gene database and MOE software from ImMunoGeneTics (IMGT) via their website http://imgt.cines.fr, or from The Immunoglobulin Facts Book, 2001, ISBN 012441351.

The term “host cell” refers to a cell into which the expression vector has been introduced. Host cells may include microbial (e.g. bacterial), plant or animal cells. Bacteria that are susceptible to be transformed include members of enterobacteriaceae, such as Escherichia coli or Salmonella strains; Bacillaceae such as Bacillus subtilis; Pneumococcus; Streptococcus and Haemophilus influenzae. Suitable microorganisms include Saccharomyces cerevisiae and Pichia pastoris. Suitable animal host cell lines include, but are not limited to CHO (Chinese hamster ovary cell line), HEK cells (as non-limiting examples, HEK293E cells) and NSO cells.

The engineered antibodies or antigen binding fragments of the present disclosure may be prepared and purified using known methods. For example, cDNA sequence encoding a heavy chain and a light chain may be cloned and engineered into a GS expression vector. The recombinant immunoglobulin expression vector may then be stably transfected into CHO cells. As a more recommended method well known in the art, mammalian expression systems will result in glycosylation of the antibody, typically at highly conserved N-terminal sites in the Fc region. Stable clones may be verified for expression of an antibody specifically binding to human IL-5. Positive clones may be expanded in serum-free culture medium in bioreactors for antibody production. Culture medium, into which an antibody has been secreted, may be purified by conventional techniques. For example, purification can be performed on Protein A or G Sepharose FF column that has been equilibrated with an adjusted buffer. The column is washed to remove nonspecific binding components, and then the bound antibody is eluted by pH gradient and antibody fractions are detected by SDS-PAGE, and then collected. The antibodies may be filtered and concentrated using common techniques. Soluble mixtures and polymers may be removed by common techniques, such as size exclusion or ion exchange. The resulting product is then immediately frozen, for example at −70° C., or may be lyophilized.

“Administration”, “administering” or “treatment,” as it applies to an animal, human, subject, cell, tissue, organ, or biological fluid, refers to contacting an exogenous pharmaceutical, therapeutic, diagnostic agent, or composition with the animal, human, subject, cell, tissue, organ, or biological fluid. “Administration”, “administering” or “treatment” can refer, e.g., to therapeutic, pharmacokinetic, diagnostic, research, and experimental methods. Treatment of a cell encompasses contacting a reagent with the cell, as well as contacting a reagent with a fluid, where the fluid is in contact with the cell. “Administration”, “administering” or “treatment” also means in vitro and ex vivo treatments of a cell, with a reagent, diagnostic, binding composition, or with another cell. “Treatment,” as it applies to a human, veterinary, or research subject, refers to therapeutic treatment, prophylactic or preventative measures, to research and diagnostic applications.

“Treat” means administration of a therapeutic agent, such as a composition containing any of antibodies or fragment thereof of the present disclosure, internally or externally to a patient having one or more disease symptom(s) for which the agent has known therapeutic activity. Typically, the agent is administered in an amount effective to alleviate one or more disease symptom(s) in the patient or population to be treated, by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present disclosure (e.g., a treatment method or article of manufacture) may not be effective in alleviating the target disease symptom(s) in every patient, it should alleviate the target disease symptom(s) in a statistically significant number of patients as determined by any statistical test known in the art such as the Student's t-test, the chi-square test, the U-test according to Mann and Whitney, the Kruskal-Wallis test (H-test), Jonckheere-Terpstra-test and the Wilcoxon-test.

“Conservative modification” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitution in non-essential regions of a polypeptide does not substantially alter biological activity (see, e.g., Watson et al. (1987) Molecular Biology of the Gene, The Benjamin/Cummings Pub. Co., p. 224 (4th Ed.)). In addition, substitutions with structurally or functionally similar amino acids are less likely to disrupt biological activity.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health condition of the patient, the route and dose of administration and the severity of side effects. An effective amount can be the maximal dose or dosing protocol that avoids significant side effects or toxic effects.

“Exogenous” refers to substances that are produced outside an organism, cell, or human body, depending on the context. “Endogenous” refers to substances that are produced within a cell, organism, or human body, depending on the context.

“Homology” refers to sequence similarity between two polynucleotide sequences or between two polypeptide sequences. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions to be compared and then multiplied by 100. For example, if 6 out of 10 positions in two sequences are matched or homologous when the sequences are optimally aligned, then the two sequences have 60% homology; if 95 out of 100 positions in two sequences are matched or homologous, then the two sequences have 95% homology. Generally, the comparison is performed when two sequences are aligned to give maximum percent homology.

As used herein, the expressions “cell,” “cell line,” and “cell culture” are used interchangeably and all such designations include progeny. Thus, the words “transformants” and “transformed cells” include the primary subject cells and cultures derived therefrom regardless of the number of passages. It should be also understood that all progeny may not be precisely identical in DNA content, due to deliberate or inadvertent mutations. Mutant progeny that have the same function or biological activity as screened in the originally transformed cells are included. Where distinct designations are intended, it will be clearly understood from the context.

As used herein, “polymerase chain reaction” or “PCR” refers to a procedure or technique in which minute amounts of a specific portion of nucleic acid, RNA and/or DNA, are amplified as described in, e.g., U.S. Pat. No. 4,683,195. Generally, sequence information about the ends of the region of interest or beyond the region of interest needs to be available, such that oligonucleotide primers can be designed; these primers will be identical or similar in sequence to opposite strands of the template to be amplified. The 5′ terminal nucleotides of the two primers are in consistence with the ends of the material to be amplified. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.). The PCR test used in the present disclosure is considered to be one, but not the only, example of polymerase reaction method for amplifying a nucleic acid test sample. The method comprises the use of known nucleic acid sequences as primers and nucleic acid polymerase to amplify or generate a specific portion of nucleic acid.

“Optional” or “optionally” means that the event or situation that follows may but does not necessarily occur, and the description includes the instances in which the event or circumstance does or does not occur. For example, “optionally contains 1-3 antibody heavy chain variable regions” means the antibody heavy chain variable region with specific sequence can be, but need not be, present.

“Pharmaceutical composition” refers to a mixture containing one or more compound(s) according to the present disclosure or a physiologically/pharmaceutically acceptable salt or produg thereof and other chemical components, such as physiologically/pharmaceutically acceptable carriers and excipients. The pharmaceutical composition aims at promoting the administration to an organism, facilitating the absorption of the active ingredient and thereby exerting a biological effect.

Furthermore, the present disclosure includes an agent for treating diseases associated with IL-5, and the agent comprises the monoclonal antibody of the present disclosure or antibody fragment thereof as an active ingredient.

There is not limitation for the diseases associated with IL-5, as long as they are associated with IL-5. For example, the therapeutic responses induced by the molecules of present disclosure can be generated by binding to human IL-5 and consequently repressing or inhibiting eosinophil-induced stimulation. Thus, when being in preparations and formulations suitable for therapeutic applications, the molecules of the present disclosure are very useful for individuals who are suffering from allergic and/or atopic responses or responses associated with eosinophils, for example, but are not limited to, asthma, asthma exacerbation, malignant attack of asthma, chronic pneumonia, allergic rhinitis, perennial allergic rhinitis, allergic bronchopulmonary aspergillosis, eosinophilia, Churg-Strauss syndrome, atopic dermatitis, onchocerciasis dermatitis, intermittent angioedema, eosinophilic myalgia syndrome, eosinophilic gastroenteritis, helminth infection, Hodgkin's disease, nasal polyps, Loeffler's syndrome, urticaria, eosinophil hyperplastic bronchitis, nodular arteritis, sinusitis, eosinophilic esophagitis, allergic eosinophilic esophagitis, allergic conjunctivitis, onchocerciasis dermatitis, endometriosis and steroid dependent eosinophilic bronchitis, and the like. In a preferred embodiment, such treatment inhibits or reduces the infiltrating-eosinophils in lung tissue. The antibodies or fragment thereof may be administered from three times a day to once every six months, and may be intravenously, subcutaneously, intramuscularly, parenterally or topically administered.

Furthermore, the present disclosure relates to an immunodetection or immunoassay method of IL-5, reagents for immunodetection or immunoassay of IL-5, an immunodetection or immunoassay of cells expressing IL-5, and a diagnostic agent for diagnosing a disease associated with IL-5, which comprises the monoclonal antibody or antibody fragment of the present disclosure specifically recognizing human IL-5 and binding to the amino acid sequence of the extracellular region or the three-dimensional structure thereof as an active ingredient.

In the present disclosure, the method for detecting or determining the amount of IL-5 may be any method known in the art. For example, it includes immunodetection or immunoassay.

The immunodetection or immunoassay is a method for detecting or determining the amount of an antibody or of an antigen by using a labeled antigen or antibody. Examples of immunodetection or immunoassay include radioactive substance labeling immunological antibody method (RIA), enzyme immunoassay (EIA or ELISA), fluorescent immunoassay (FIA), luminescent immunoassay, Western Blotting, physicochemical assays, and the like.

The above diseases associated with IL-5 can be diagnosed by detecting or determining the cells expressing IL-5 with the monoclonal antibody or antibody fragment of the present disclosure.

In order to detect cells expressing the polypeptide, a known immunoassay can be used, preferably, immunoprecipitation, fluorescent cell staining, immunohistochemical staining, and the like is used. Further, a fluorescent antibody staining method with FMAT8100HTS system (Applied Biosystem), and the like can be used.

In the present disclosure, a living sample used for detecting or determining IL-5 is not particularly limited, as long as it is likely to contain cells expressing IL-5, for example, tissue cells, blood, plasma, serum, pancreatic fluid, urine, feces, tissue fluid or culture medium can be used.

The diagnostic agent comprising the monoclonal antibody or antibody fragment thereof of the present disclosure may further comprise an agent for performing antigen-antibody reaction or an agent for detecting the reaction, depending on a desired diagnostic method. The agent for performing antigen-antibody reaction includes such as buffer and salts. The agent for detecting the reaction includes reagents commonly used in immunodetection or immunoassay method, for example, such as a labeled secondary antibody recognizing the monoclonal antibody, antibody fragment thereof or conjugate comprising the same, and a substrate corresponding to the labels.

### 2. Examples and Test Examples

The following examples are provided to further describe the present disclosure, but are not intended to limit the scope of the disclosure. Experimental methods for which the specific conditions are not specifically indicated are generally carried out according to conventional conditions, see Molecular Cloning, Laboratory Manual of antibody technology, Cold Spring Harbor Laboratory; or according to the conditions recommended by the manufacturer of materials or products. Reagents for which the sources are not specifically indicated are commercially available reagents.

### Example 1. Preparation of IL-5 Antigens and Proteins for Detection

### Design and Expression of IL-5 Antigen

Sequences encoding His-tagged human IL-5, Rhesus monkey IL-5, mouse IL-5, rat IL-5, or human IL-5Rα receptor extracellular domain fused to human IgG1-Fc fragment were inserted into the phr vector, to construct expression plasmids, which were then transfected into HEK293. On day 6 after transfection, samples were centrifuged at 4500 rpm for 10 min and cell supernatants were collected. The supernatant containing recombinant IL-5 or IL-5α receptor protein was purified by using nickel column, and the recombinant human IL-5-Fc fusion protein was purified by using Protein A affinity chromatography column. The purified protein can be used in the following examples. The protein sequences of the antigen are shown as follows:

1. Human IL-5 amino acid sequence with his tag
(rhIL-5-his)
SEQ ID NO:1
MRMLLHLSLLALGAAYVYAIPTEIPTSALVKETLALLSTHRTLLIANETL
RIPVPVHKNHQLCTEEIFQGIGTLESQTVQGGTVERLFKNLSLIKKYIDG
QKKKCGEERRRVNQFLDYLQEFLGVMNTEWIIESHHHHHH
Note: The italic text shows His6-tag
2. Cynomolgus monkey IL-5 amino acid sequence with
his tag
SEQ ID NO: 2
MRMLLHLSLLALGAAYVYAIPTEIPASALVKETLALLSTHRTLLIANETL
RIPVPVHKNHQLCTEEIFQGIGTLESQTVQGGTVERLFKNLSLIKKYIGG
QKKKCGEERRRVNQFLDYLQEFLGVMNTEWIIESHHHHHH
Note: The italic text shows His6-tag.
3. Mouse IL-5 amino acid sequence with his tag
SEQ ID NO: 3
MEIPMSTVVKETLTQLSAHRALLTSNETMRLPVPTHKNHQLCIGEIFQGL
DILKNQTVRGGTVEMLFQNLSLIKKYIDRQKEKCGEERRRTRQFLDYLQE
FLGVMSTEWAMEGHHHHHH
Note: The italic text shows His6-tag.
4. Rat IL-5 amino acid sequence with his tag
SEQ ID NO: 4
MEIPMSTVVKETLIQLSTHRALLTSNETMRLPVPTHKNHQLCIGEIFQGL
DILKNQTVRGGTVEILFQNLSLIKKYIDGQKEKCGEERRKTRHFLDYLQE
FLGVMSTEWAMEVHHHHHH
Note: The italic text shows His6-tag.
5. Amino acid sequence of human IL-5α receptor
fused to human Fc fragment
SEQ ID NO: 5
DLLPDEKISLLPPVNFTIKVTGLAQVLLQWKPNPDQEQRNVNLEYQVKIN
APKEDDYETRITESKCVTILHKGFSASVRTILQNDHSLLASSWASAELHA
PPGSPGTSIVNLTCTTNTTEDNYSRLRSYQVSLHCTWLVGTDAPEDTQYF
LYYRYGSWTEECQEYSKDTLGRNIACWFPRTFILSKGRDWLAVLVNGSSK
HSAIRPFDQLFALHAIDQINPPLNVTAEIEGTRLSIQWEKPVSAFPIHCF
DYEVKIHNTRNGYLQIEKLMTNAFISIIDDLSKYDVQVRAAVSSMCREAG
LWSEWSQPIYVGNDEHKPLREWIEGRMDEPKSCDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPGK
Note: The italic text shows human Fc tag.

### Example 2: Construction and Identification of Recombinant IL-5α Receptor and IL-5α/β Receptor Cell Lines

To screen for functional antibodies, the present disclosure construct a CHO-S/IL-5α cell line expressing IL-5α, and a CHO-S/IL-5α/IL-5B cell line expressing both IL-5α and IL-5β.

Specifically, full-length human IL-5α gene (Q01344) was cloned into a mammalian cell expression vector, pTargeT, and the linearized plasmid was transfected into CHO-S cells via electroporation. Screening was performed under G418 for 2 weeks, followed by two rounds of limiting dilutions. The IL-5α gene expressed on cell surface was detected by FACS, CHO-S/IL-5α cell lines with high expression level of IL-5α were selected and transfected into the linearized pcDNA3.1-IL-5B via electroporation. Screening was performed with G418 and zeocin for 2 weeks, followed by two rounds of limiting dilutions. The IL-5α and IL-5β gene expressed on the cell surface were detected by FACS, and CHO-S/IL-5α/IL-5β cell lines with high expression of IL-5α and IL-5ß were selected.

### Example 3: Preparation of Anti-Human IL-5 Murine Monoclonal Antibody

Two groups of Balb/c mice (5 mice/group) and four groups of SJL mice (5 mice/group) were immunized with recombinant protein rhIL-5-his and Freund's adjuvant CFA (Sigma, Lot #SLBQ1109V); or with IFA (Sigma, Lot #SLBJ2845V) at two dosages of 100 g/50 g/50 g (high-dose group) and of 25 g/12.5 g/12.5 g (low-dose group), respectively. The specific immune response to IL-5 was determined by detecting serum titer by ELISA, Ligand-Receptor Blocking assay and TF-1 Proliferation Inhibition Assay. Mice with better specific immune response were selected, sacrificed, spleen cells were collected and fused with myeloma cells.

Primary screening was performed by using an ELISA binding assay against human IL-5. The hybridoma cells were transferred into 24-well plates, and the supernatants were rescreened by ELISA binding assay against human, cynomolgus, or mouse IL-5 by ELISA-based blocking assay against IL-5 receptor and by TF-1 proliferation inhibition assay. After such screening, the obtained positive clones were subjected to two rounds of subcloning to obtain hybridoma clones for antibody production. The obtained antibodies were purified by affinity chromatography.

The purified antibodies were subjected to the following tests: SEC-HPLC, detection of endotoxin content, Biacore assay for affinity to various IL-5, FACS-based blocking assay against IL-5 receptor, and TF-1 proliferation inhibition assay, adhesion test of eosinophils, and evaluation of efficacy on mouse asthma model and in vivo guinea pig neutralization model. Monoclonal hybridoma cell lines mAb1705, mAb1706, mAb1780, mAb1773 and mAb1779 were selected for their excellent activities in vitro and in vivo.

The sequences were cloned from positive hybridoma as follows. The hybridoma cells in logarithmic growth phase were collected, RNAs were extracted with Trizol (Invitrogen, Cat No. 15596-018) according to the manufacturer's instruction or kit, and reverse transcription was performed with PrimeScript™ Reverse Transcriptase kit (Takara, Cat No. 2680A). The cDNAs obtained by reverse transcription were subjected to PCR amplification using mouse Ig-Primer Set (Novagen, TB326 Rev. B 0503) and the resulting products were sequenced. The amino acid sequences corresponding to DNA sequences of mAb1705, mAb1706, mAb1780, mAb1773 and mAb1779 heavy and light chain variable regions were obtained (the amino acid residues of the VH/VL CDRs were determined and annotated by the Kabat numbering system):

mAb1705 murine heavy chain variable region sequence
SEQ ID NO: 6
EVQLVESGGGLVQPGRSLKLSCTASGFTFSHYYMAWVRQAPKKGLEWVTSISY
EGDITYYGDSVKGRFTISRDNAKSTLYLQMNSLRSEDTATYYCASQTLRESFDY
WGQGVMVTVSS
mAb1705 murine light chain variable region sequence
SEQ ID NO: 7
DIQMTQSPSSMSVSLGDRVTITCRASQDIANYLSWYQQKIARSPKLVIYGTS
NLEVGVPSRFSGSRSGSDYSLTINTLESEDTGIYFCLQDKEFPRTFGGGTRLE
LK
mAb1706 murine heavy chain variable region sequence
SEQ ID NO: 8
EVQLVESGGGLVQPGRSLKLSCAASGFTFSHYYMAWVRQAPKKGLEWVTSINY
EGNSAYYGDSVKGRFTISRDNAKSTLYLQMDSLRSEDTATYYCATETLRESLDY
WGQGVMVTVSS
mAb1706 murine light chain variable region sequence
SEQ ID NO: 9
DIQMTQSPSSMSVSLGDRVTITCRASQDIGNYLSWYQQKLGKSPKLMIHSA
SNLEVGVPSRFSGSRSGSDYSLTINTLESEDPGIYFCLQHKQFPRTFGGGTKL
ELK
mAb1780 murine heavy chain variable region sequence
SEQ ID NO:10
QVKLLQSGAALVKPGDSVKMSCKASDYTFTEYLIHWVKQSQGRSLEWIGYINP
YSGGTVYNEKFKSKATLTVDKFSSTAYMEFRRLTFEDSAIYYCARDGGYSDPLD
YWGQGVMVTVSS
mAb1780 murine light chain variable region sequence
SEQ ID NO:11
DTVLTQSPALAVSPGERVSISCRASEGLTSYMHWYQQKPGQQPKLLIYKASNLA
SGVPARFSGSGSGTDFTLTIDPVEADDAATYFCQQNWNDPWTFGGGTKLELK
mAb1773 murine heavy chain variable region sequence
SEQ ID NO:12
EVQLQQSLAELVRPGASVTLSCTASGFNIKNTYIHWVKQRPEQGLEWIGRIDPA
NGDTKHGPKFQGKATITADTSSNTAYLQFSSLTSEDTAIYYCFRYGIYPDHWGQG
TPLTVSS
mAb1773 murine light chain variable region sequence
SEQ ID NO:13
QIVLTQSPALMSASPGEKVTMTCSASSSVNYIYWYQQKPRSSPKPWIYLTATLAS
GVPARFSGSGSGTSFSLTISRMEAEDAATYYCQQWNSYPYTFGGGTKLEIE
mAb1779 murine heavy chain variable region sequence
SEQ ID NO:14
QVKLLQSGAALVKPGDSVKMSCKASGYTFTDYIIHWVKQSHGKSLEWIGYFNP
NSGGSNYNENFKRKATLTADKSSSTAYLEFSRVTSEDSAIYYCGRRIAWDHWYF
DFWGPGTMVTVSS
mAb1779 murine light chain variable region sequence
SEQ ID NO:15
DIQMTQSPASLSASLGETVSIECLASEGISNDVAWYQQKSGRSPQLLVYAASRLQ
DGVPSRFSGSGSGTRYFFKISGMQPEDEADYFCQQGYKTPLTFGSGTKLEIK

The light and heavy chain CDR sequences of each antibody are shown in Table 1.

TABLE 1
Heavy and light chain CDR sequences of each
antibody

| Antibody | Heavy chain | | Light chain | |
|---|---|---|---|---|
| mAb1705 | HCDR1 | HYYMA | LCDR1 | RASQDIANYLS |
| | | SEQ ID NO:16 | | SEQ ID NO:19 |
| | HCDR2 | SISYEGDITYYGD | LCDR2 | GTSNLEV |
| | | SVKG | | SEQ ID NO: 20 |
| | | SEQ ID NO:17 | | |
| | HCDR3 | QTLRESFDY | LCDR3 | LQDKEFPRT |
| | | SEQ ID NO:18 | | SEQ ID NO: 21 |
| mAb1706 | HCDR1 | HYYMA | LCDR1 | RASQDIGNYLS |
| | | SEQ ID NO: 22 | | SEQ ID NO: 25 |
| | HCDR2 | SINYEGNSAYYGD | LCDR2 | SASNLEV |
| | | SVKG | | SEQ ID NO: 26 |
| | | SEQ ID NO: 23 | | |
| | HCDR3 | ETLRESLDY | LCDR3 | LQHKQFPRT |
| | | SEQ ID NO: 24 | | SEQ ID NO: 27 |
| mAb1780 | HCDR1 | EYLIH | LCDR1 | RASEGLTSYMH |
| | | SEQ ID NO: 28 | | SEQ ID NO: 31 |
| | HCDR2 | YINPYSGGTVYNE | LCDR2 | KASNLAS |
| | | KFKS | | SEQ ID NO: 32 |
| | | SEQ ID NO: 29 | | |
| | HCDR3 | DGGYSDPLDY | LCDR3 | QQNWNDPWT |
| | | SEQ ID NO: 30 | | SEQ ID NO: 33 |
| mAb1773 | HCDR1 | NTYIH | LCDR1 | SASSSVNYIY |
| | | SEQ ID NO: 34 | | SEQ ID NO: 37 |
| | HCDR2 | RIDPANGDTKHGP | LCDR2 | LTATLAS |
| | | KFQG | | SEQ ID NO: 38 |
| | | SEQ ID NO: 35 | | |
| | HCDR3 | YGIYPDH | LCDR3 | QQWNSYPYT |
| | | SEQ ID NO: 36 | | SEQ ID NO: 39 |
| mAb1779 | HCDR1 | DYIIH | LCDR | LASEGISNDVA |
| | | SEQ ID NO: 40 | | SEQ ID NO: 43 |
| | HCDR2 | YFNPNSGGSNYNE | LCDR2 | AASRLQD |
| | | NFKR | | SEQ ID NO: 44 |
| | | SEQ ID NO: 41 | | |
| | HCDR3 | RIAWDHWYFDF | LCDR3 | QQGYKTPLT |
| | | SEQ ID NO: 42 | | SEQ ID NO: 45 |

The activity results of Biacore assay are shown in Table 2.

TABLE 2
In vitro activity of IL-5 murine antibody

| | affinity to HuIL-5 |
|---|---|
| antibody | (KD (M)) |
| mAb1705 | 7.27E−11 |
| mAb1706 | 3.83E−11 |
| mAb1780 | 8.99E−11 |
| mAb1773 | 1.29E−10 |
| mAb1779 | 4.58E−10 |

The results show that the murine antibodies of the present disclosure have high affinity to the antigen.

### Example 4: Purification of IL-5-Related Recombinant Proteins, and Purification of Hybridoma Antibodies and Recombinant Antibodies

### 4.1 Steps for Purification of IL-5-Flag-his Recombinant Proteins:

Samples were centrifuged at high speed to remove impurities and concentrated to appropriate volume. The NI-NTA affinity column (QIAGEN, Cat No. 30721) was equilibrated with PBS and washed with 2-5 column volumes. The cell-expressed supernatants without impurities were loaded onto the column, which was then rinsed with PBS until the A280 reading was dropped to the baseline. Then the column was rinsed with PBS to remove the impure protein. The protein of interest was eluted with washing buffer (imidazole 20 mM) and then elution buffer (imidazole 300 mM), and the eluted peak was collected.

The collected elute was further purified by ion exchange (Hiload 16/600 superdex 200 column). The column was equilibrated with about 2 column volumes of PBS to ensure pH of 7.4. The elution buffer containing the protein of interest was concentrated and loaded onto the column for subsequent purification, the samples were collected, identified by SDS-PAGE and LC-MS, and then aliquoted for use.

### 4.2. Purification of Hybridoma-Expressed Antibody and Fc Fusion Proteins

The cell-expressed supernatant samples were centrifuged at high speed to remove impurities, and then the hybridoma-expressed supernatants were purified by using Protein G column, the Fc fusion protein expressing supernatants were purified by using Protein A column. The column was rinsed with PBS until the A280 reading was dropped to the baseline. The proteins of interest were eluted with 100 mM acetic acid, pH 3.0 and neutralized with 1 M Tris-HCl, pH 8.0. The eluted samples were appropriately concentrated and further purified by gel chromatography Superdex 200 (GE) pre-equilibrated with PBS. The peaks deprived of aggregates were collected and aliquoted for use.

### Example 5: Humanization Design of Anti-Human IL-5 Monoclonal Antibodies

Humanization of murine anti-human IL-5 monoclonal antibodies was carried out as disclosed in literatures in the art. Briefly, the constant regions of murine antibodies were replaced with human constant regions, the CDRs of murine antibodies were grafted onto the human template sharing the highest FR homology, and some amino acid residues in FR region, which have key effect on maintaining the antibody conformation and affecting the binding of the antibody to the antigen, were back-mutated.

By aligning to IMGT human antibody heavy and light chain variable region germline gene database, human germline heavy and light chain variable region genes, which have high identity to amino acid sequence of mAb-1705, mAb-1706, mAb1780, mAb1773 and mAb1779 antibody, were selected as templates respectively. The CDRs of these murine antibodies were separately grafted onto the corresponding human derived templates to form a variable region sequence in the order of FRI-CDR1-FR2-CDR2-FR3-CDR3-FR4. The amino acid residues were determined and annotated by the Kabat numbering system.

### Selection of Human FR Region and Back Mutation of Key Amino Acids

Based on the typical VH/VL CDR structure of the obtained murine antibody, homologous sequences of light chain variable region (VL) and heavy chain variable region (VH) was selected from the human germline database. The resulting human germline VL and VH sequences were ranked from high to low, based on FR homology, and the germline sequences with the highest FR homology were selected as main templates. The CDRs of the murine antibodies were grafted onto the human templates. And then by using software and based on the three-dimensional structure of the murine antibodies, the embedded residues, residues which interact directly with the CDR regions, and residues which have significant effects on the conformation of VL and VH were subjected to back mutations. Further, chemically unstable amino acid residues were optimized to yield final humanized molecules.

5.1 Selection of Humanized Framework for Hybridoma Clone mAb1705

IGHV3-23*04 was selected as the template for h1705 VH, and IGKV1-12*01 was selected as the template for VL. The CDRs of murine mAb 1705 were grafted onto the human template. The embedded residues and residues which interact directly with the CDR regions were found by software and were subjected to back-mutation. The light and heavy chain variable regions of the humanized antibodies were designed as shown in Table 3.

TABLE 3
Selection of template and design of back mutation for h1705

| h1705_VL | | h1705_VH | |
|---|---|---|---|
| h1705_VL.1 | Grafted | h1705_VH.1 | Grafted |
| h1705_VL.1A | A43S, G66R | h1705_VH.1A | K98S |
| h1705_VL.1B | A43S, L47V, | h1705_VH.1B | S49T, V93T, |
| | G66R, T69S, | | K98S |
| | F71Y, Y87F | | |

Note:
“Grafted” means the murine antibody CDRs were grafted onto the human germline FR region sequence. For example, A43S indicates that A on position 43 of the grafted sequence was back-mutated to S, according to the natural sequence numbering of the amino acid sequence.

TABLE 4
Combination of h1705 humanized antibody
heavy and light chain variable region

| | h1705_VH.1 | h1705_VH.1A | h1705_VH.1B |
|---|---|---|---|
| h1705_VL.1 | h1705-003 | h1705-004 | h1705-005 |
| h1705_VL.1A | h1705-006 | h1705-007 | h1705-008 |
| h1705_VL.1B | h1705-009 | h1705-010 | h1705-011 |

Note:
This table shows sequences obtained by combining various variants. For example, h1705-007 indicates that the humanized murine antibody h1705-007 comprises two variants, i.e., light chain h1705_VL.1A and heavy chain h1705_VH.1A, and so forth.

The specific sequence of variable regions of humanized antibody h1705 are as follows:

> h1705_VL.1
(SEQ ID NO: 46)
DIQMTQSPSSVSASVGDRVTITCRASQDIANYLSWYQQKPGKAPKLLIYG
TSNLEVGVPSRFSGSGSGTDFTLTISSLQPEDFATYYCLQDKEFPRTFGG
GTKVEIK
> h1705_VL.1A
(SEQ ID NO: 47)
DIQMTQSPSSVSASVGDRVTITCRASQDIANYLSWYQQKPGKSPKLLIYG
TSNLEVGVPSRFSGSRSGTDFTLTISSLQPEDFATYYCLQDKEFPRTFGG
GTKVEIK
> h1705_VL.1B
(SEQ ID NO: 48)
DIQMTQSPSSVSASVGDRVTITCRASQDIANYLSWYQQKPGKSPKLVIYG
TSNLEVGVPSRFSGSRSGSDYTLTISSLQPEDFATYFCLQDKEFPRTFGG
GTKVEIK
> h1705_VH.1
(SEQ ID NO: 49)
EVQLVESGGGLVQPGGSLRLSCAASGFTFSHYYMAWVRQAPGKGLEWVSS
ISYEGDITYYGDSVKGRFTISRDNSKNTLYLQMNSLRAEDTAVYYCAKQT
LRESFDYWGQGTLVTVSS
> h1705_VH.1A
(SEQ ID NO: 50)
EVQLVESGGGLVQPGGSLRLSCAASGFTFSHYYMAWVRQAPGKGLEWVSS
ISYEGDITYYGDSVKGRFTISRDNSKNTLYLQMNSLRAEDTAVYYCASQT
LRESFDYWGQGTLVTVSS
> h1705_VH.1B
(SEQ ID NO: 51)
EVQLVESGGGLVQPGGSLRLSCAASGFTFSHYYMAWVRQAPGKGLEWVTS
ISYEGDITYYGDSVKGRFTISRDNSKNTLYLQMNSLRAEDTATYYCASQT
LRESFDYWGQGTLVTVSS

Each of the above light chain variable regions was combined with light chain constant region as set forth in SEQ ID NO: 53 to form the final intact light chain sequences. Each heavy chain variable region was combined with heavy chain constant region as set forth in SEQ ID NO: 52 to form the final heavy chain sequences.

### Humanized Antibody Constant Region Sequence

>Heavy chain IgGl constant region:
SEQ ID NO: 52
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Note: Underlined text represents the designed
M252Y, S254T or T256E mutation.
>Light chain kappa constant region:
SEQ ID NO: 53
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

5.2 Selection of Humanized Framework for Hybridoma Clone mAb1706

IGHV3-23*04 was selected as the template for h1706 VH, and IGKV1-12*01 was selected as the template for VL. The CDRs of murine mab 1706 were grafted onto the human template. The embedded residues and residues which interact directly with the CDR regions were found by software and were subjected to back-mutation. The light and heavy chain variable regions of the humanized antibodies were designed as shown in Table 5.

TABLE 5
Selection of template and design of back mutation for h1706

| h1706_VL | | h1706_VH | |
|---|---|---|---|
| h1706_VL.1 | Grafted | h1706_VH.1 | Grafted |
| h1706_VL.1A | A43S | h1706_VH.1A | K98T |
| h1706_VL.1B | A43S, L47M, | h1706_VH.1B | S49T, V93T, |
| | F71Y, Y87F | | K98T |

Note:
“Grafted” means the murine antibody CDRs were grafted onto the human germline FR region. For example, A43S indicates that A on position 43 of the grafted sequence was back-mutated to S, according to the natural sequence numbering of the amino acid sequence.

TABLE 6
Combination of h1706 humanized antibody
heavy and light chain variable region

| | h1706_VH.1 | h1706_VH.1A | h1706_VH.1B |
|---|---|---|---|
| h1706_VL.1 | h1706-002 | h1706-003 | h1706-004 |
| h1706_VL.1A | h1706-005 | h1706-006 | h1706-007 |
| h1706_VL.1B | h1706-008 | h1706-009 | h1706-010 |

The specific sequences of the variable regions of humanized antibody h1706 are shown as follows:

> h1706_VL.1
(SEQ ID NO: 54)
DIQMTQSPSSVSASVGDRVTITCRASQDIGNYLSWYQQKPGKAPKLLIYS
ASNLEVGVPSRFSGSGSGTDFTLTISSLQPEDFATYYCLQHKQFPRTFGG
GTKVEIK
> h1706_VL.1A
(SEQ ID NO: 55)
DIQMTQSPSSVSASVGDRVTITCRASQDIGNYLSWYQQKPGKSPKLLIYS
ASNLEVGVPSRFSGSGSGTDFTLTISSLQPEDFATYYCLQHKQFPRTFGG
GTKVEIK
> h1706_VL.1B
(SEQ ID NO: 56)
DIQMTQSPSSVSASVGDRVTITCRASQDIGNYLSWYQQKPGKSPKLMIYS
ASNLEVGVPSRFSGSGSGTDYTLTISSLQPEDFATYFCLQHKQFPRTFGG
GTKVEIK
> h1706_VH.1
(SEQ ID NO: 57)
EVQLVESGGGLVQPGGSLRLSCAASGFTFSHYYMAWVRQAPGKGLEWVSS
INYEGNSAYYGDSVKGRFTISRDNSKNTLYLQMNSLRAEDTAVYYCAKET
LRESLDYWGQGTMVTVSS
> h1706_VH.1A
(SEQ ID NO: 58)
EVQLVESGGGLVQPGGSLRLSCAASGFTFSHYYMAWVRQAPGKGLEWVSS
INYEGNSAYYGDSVKGRFTISRDNSKNTLYLQMNSLRAEDTAVYYCATET
LRESLDYWGQGTMVTVSS
> h1706_VH.1B
(SEQ ID NO: 59)
EVQLVESGGGLVQPGGSLRLSCAASGFTFSHYYMAWVRQAPGKGLEWVTS
INYEGNSAYYGDSVKGRFTISRDNSKNTLYLQMNSLRAEDTATYYCATET
LRESLDYWGQGTMVTVSS

Each of the above light chain variable regions was combined with light chain constant region as set forth in SEQ ID NO: 53 to form the final intact light chain sequences. Each heavy chain variable region was combined with heavy chain constant region as set forth in SEQ ID NO: 52 to form the final heavy chain sequences.

5.3 Selection of Humanized Framework for Hybridoma Clone mAb1780

IGHV1-2*02 was selected as the template for h1780 VH, and IGKV3-11*01 was selected as the template for VL. The CDRs of murine mAb 1780 were grafted onto the human template. The embedded residues and residues which interact directly with the CDR regions were found by software and were subjected to back-mutation. The light and heavy chain variable regions of the humanized antibodies were designed as shown in Table 7.

TABLE 7
Selection of template and design of back mutation for h1780

| h1780_VL | | h1780_VH | |
|---|---|---|---|
| h1780_VL.1 | Grafted | h1780_VH.1 | Grafted |
| h1780_VL.1A | E1D, I2T | h1780_VH.1A | M70L, R72V, T74K |
| h1780_VL.1B | E1D, I2T, | h1780_VH.1B | M48I, V68A, M70L, |
| | I57V, V84T, | | R72V, T74K, L83F |
| | Y86F | | |
| | | h1780_VH.1C | R38K, M48I, R67K, |
| | | | V68A, M70L, R72V, |
| | | | T74K, L83F |

Note:
“Grafted” means the murine antibody CDRs were grafted onto the human germline FR region. For example, E1D indicates that E on position 1 of the grafted sequence was back-mutated to D, according to the natural sequence numbering of the amino acid sequence.

TABLE 8
Combination of h1780 humanized antibody heavy and light chain variable region

| | h1780_VH.1 | h1780_VH.1A | h1780_VH.1B | h1780_VH.1C |
|---|---|---|---|---|
| h1780_VL.1 | h1780-007 | h1780-008 | h1780-009 | h1780-010 |
| h1780_VL.1A | h1780-011 | h1780-012 | h1780-013 | h1780-014 |
| h1780_VL.1B | h1780-015 | h1780-016 | h1780-017 | h1780-018 |

The specific sequences of the variable regions of humanized antibody h1780 are shown as follows:

> h1780_VL.1
(SEQ ID NO: 60)
EIVLTQSPATLSLSPGERATLSCRASEGLTSYMHWYQQKPGQAPRLLIYK
ASNLASGIPARFSGSGSGTDFTLTISSLEPEDFAVYYCQQNWNDPWTFGG
GTKVEIK
> h1780_VL.1A
(SEQ ID NO: 61)
DTVLTQSPATLSLSPGERATLSCRASEGLTSYMHWYQQKPGQAPRLLIYK
ASNLASGIPARFSGSGSGTDFTLTISSLEPEDFAVYYCQQNWNDPWTFGG
GTKVEIK
> h1780_VL.1B
(SEQ ID NO: 62)
DTVLTQSPATLSLSPGERATLSCRASEGLTSYMHWYQQKPGQAPRLLIYK
ASNLASGVPARFSGSGSGTDFTLTISSLEPEDFATYFCQQNWNDPWTFGG
GTKVEIK
> h1780_VH.1
(SEQ ID NO: 63)
EVQLVQSGAEVKKPGASVKVSCKASGYTFTEYLIHWVRQAPGQGLEWMGY
INPYSGGTVYNEKFKSRVTMTRDTSISTAYMELSRLRSDDTAVYYCARDG
GYSDPLDYWGQGTMVTVSS
> h1780_VH.1A
(SEQ ID NO: 64)
EVQLVQSGAEVKKPGASVKVSCKASGYTFTEYLIHWVRQAPGQGLEWMGY
INPYSGGTVYNEKFKSRVTLTVDKSISTAYMELSRLRSDDTAVYYCARDG
GYSDPLDYWGQGTMVTVSS
> h1780_VH.1B
(SEQ ID NO: 65)
EVQLVQSGAEVKKPGASVKVSCKASGYTFTEYLIHWVRQAPGQGLEWIGY
INPYSGGTVYNEKFKSRATLTVDKSISTAYMEFSRLRSDDTAVYYCARDG
GYSDPLDYWGQGTMVTVSS
> h1780_VH.1C
(SEQ ID NO: 66)
EVQLVQSGAEVKKPGASVKVSCKASGYTFTEYLIHWVKQAPGQGLEWIGY
INPYSGGTVYNEKFKSKATLTVDKSISTAYMEFSRLRSDDTAVYYCARDG
GYSDPLDYWGQGTMVTVSS

Each of the above light chain variable regions was combined with light chain constant region as set forth in SEQ ID NO: 53 to form the final intact light chain sequences. Each heavy chain variable region was combined with heavy chain constant region as set forth in SEQ ID NO: 52 to form the final heavy chain sequences.

5.4 Selection of Humanized Framework for Hybridoma Clone mAb1773

IGHV3-73*01 was selected as the template for h1773 VH, and IGKV1-39*01 was selected as the template for VL. The CDRs of murine mAb1773 were grafted onto the human template. The embedded residues and residues which interact directly with the CDR regions were found by software and were subjected to back-mutation. The light and heavy chain variable regions of the humanized antibodies were designed as shown in Table 9. Meanwhile, in order to eliminate isomerized sites in CDR regions, N located in HCDR2 (RIDPANGDTK HGPKFQG) of h1773 was replaced with V (i.e., N55V) to form a heavy chain variable region and an antibody comprising HCDR2 variant (the sequence of the mutated HCDR2 is shown as SEQ ID NO: 82: RIDPAVGDTKHGPKFQG).

TABLE 9
Selection of template and design of back mutation for h1773

| h1773_VL | | h1773_VH | |
|---|---|---|---|
| h1773_VL.1 | Grafted | h1773_VH.1 | Grafted |
| h1773_VL.1A | M4L, A42S, | h1773_VH.1A | F29I, R72A, |
| | L45P, L46W | | T97F + N55V |
| | | h1773_VH.1B | F29I, R38K, |
| | | | V48I, R72A, |
| | | | T97F + N55V |

Note:
“Grafted” means the murine antibody CDRs were grafted onto the human germline FR region. For example, M4L indicates that M on position 4 of the grafted sequence was back-mutated to L, according to the natural sequence numbering of the ammo acid sequence.

TABLE 10
Combination of h1773 humanized antibody
heavy and light chain variable region

| | h1773_VH.1 | h1773_VH.1A | h1773_VH.1B |
|---|---|---|---|
| h1773_VL.1 | h1773-002 | h1773-003 | h1773-004 |
| h1773_VL.1A | h1773-005 | h1773-006 | h1773-007 |

The specific sequences of the variable regions of humanized antibody h1773 are shown as follows:

> h1773_VL.1
(SEQ ID NO: 67)
DIQMTQSPSSLSASVGDRVTITCSASSSVNYIYWYQQKPGKAPKLLIYLT
ATLASGVPSRFSGSGSGTDFTLTISSLQPEDFATYYCQQWNSYPYTFGGG
TKVEIK
> h1773_VL.1A
(SEQ ID NO: 68)
DIQLTQSPSSLSASVGDRVTITCSASSSVNYIYWYQQKPGKSPKPWIYLT
ATLASGVPSRFSGSGSGTDFTLTISSLQPEDFATYYCQQWNSYPYTFGGG
TKVEIK
> h1773_VH.1
(SEQ ID NO: 69)
EVQLVESGGGLVQPGGSLKLSCAASGFTFSNTYIHWVRQASGKGLEWVGR
IDPAVGDTKHGPKFQGRFTISRDDSKNTAYLQMNSLKTEDTAVYYCTRYG
IYPDHWGQGTLVTVSS
> h1773_VH.1A
(SEQ ID NO: 70)
EVQLVESGGGLVQPGGSLKLSCAASGFTISNTYIHWVRQASGKGLEWVGR
IDPAVGDTKHGPKFQGRFTISADDSKNTAYLQMNSLKTEDTAVYYCFRYG
IYPDHWGQGTLVTVSS
> h1773_VH.1B
(SEQ ID NO: 71)
EVQLVESGGGLVQPGGSLKLSCAASGFTISNTYIHWVKQASGKGLEWIGR
IDPAVGDTKHGPKFQGRFTISADDSKNTAYLQMNSLKTEDTAVYYCFRYG
IYPDHWGQGTLVTVSS

Each of the above light chain variable regions was combined with light chain constant region as set forth in SEQ ID NO: 53 to form the final intact light chain sequences. Each heavy chain variable region was combined with heavy chain constant region as set forth in SEQ ID NO: 52 to form the final heavy chain sequences.

5.5 Selection of Humanized Framework for Hybridoma Clone mAb1779

IGHV1-2*02 was selected as the template for h1779 VH, and IGKV1-33*01 was selected as the template for VL. The CDRs of murine h1779 were grafted onto the human template. The embedded residues and residues which interact directly with the CDR regions were found by software and were subjected to back-mutation. The light and heavy chain variable regions of the humanized antibodies were designed as shown in Table 11.

TABLE 11
Selection of template and design of back mutation for h1779

| h1779_VL | | h1779_VH | |
|---|---|---|---|
| h1779_VL.1 | Grafted | h1779_VH.1 | Grafted + D89E |
| h1779_VL.1A | A43S | h1779_VH.1A | R72A, T74K + D89E |
| h1779_VL.1B | A43S, | h1779_VH.1B | M48I, V68A, R72A, |
| | I48V, | | T74K + D89E |
| | F71Y | | |
| | | h1779_VH.1C | M48I, V68A, R72A, |
| | | | T74K, M81L, |
| | | | L83F + D89E |
| | | h1779_VH.1D | R38K, M48I, R67K, |
| | | | V68A, R72A, T74K, |
| | | | M81L, L83F + D89E |

Note:
“Grafted” means the murine antibody CDRs were grafted onto the human germline FR region. For example, A43S indicates that A on position 43 of the grafted sequence was back-mutated to S, according to the natural sequence numbering of the amino acid sequence.

The designed humanized molecules were combined to various molecules as indicated in Table 12.

TABLE 12
Combination of h1779 humanized antibody heavy and light chain variable region

| | h1779_VH.1 | h1779_VH.1A | h1779_VH.1B | h1779_VH.1C | h1779_VH.1D |
|---|---|---|---|---|---|
| h1779_VL.1 | h1779-005 | h1779-006 | h1779-007 | h1779-008 | h1779-009 |
| h1779_VL.1A | h1779-010 | h1779-011 | h1779-012 | h1779-013 | h1779-014 |
| h1779_VL.1B | h1779-015 | h1779-016 | h1779-017 | h1779-018 | h1779-019 |

The specific sequences of the variable regions of humanized antibody h1779 are shown as follows:

> h1779_VL.1
(SEQ ID NO: 72)
DIQMTQSPSSLSASVGDRVTITCLASEGISNDVAWYQQKPGKAPKLLIYA
ASRLQDGVPSRFSGSGSGTDFTFTISSLQPEDIATYYCQQGYKTPLTFGQ
GTKLEIK
> h1779_VL.1A
(SEQ ID NO: 73)
DIQMTQSPSSLSASVGDRVTITCLASEGISNDVAWYQQKPGKSPKLLIYA
ASRLQDGVPSRFSGSGSGTDFTFTISSLQPEDIATYYCQQGYKTPLTFGQ
GTKLEIK
> h1779_VL.1B
(SEQ ID NO: 74)
DIQMTQSPSSLSASVGDRVTITCLASEGISNDVAWYQQKPGKSPKLLVYA
ASRLQDGVPSRFSGSGSGTDYTFTISSLQPEDIATYYCQQGYKTPLTFGQ
GTKLEIK
> h1779_VH.1
(SEQ ID NO: 75)
EVQLVQSGAEVKKPGASVKVSCKASGYTFTDYIIHWVRQAPGQGLEWMGY
FNPNSGGSNYNENFKRRVTMTRDTSISTAYMELSRLRSEDTAVYYCARRI
AWDHWYFDFWGQGTMVTVSS
> h1779_VH.1A
(SEQ ID NO: 76)
EVQLVQSGAEVKKPGASVKVSCKASGYTFTDYIIHWVRQAPGQGLEWMGY
FNPNSGGSNYNENFKRRVTMTADKSISTAYMELSRLRSEDTAVYYCARRI
AWDHWYFDFWGQGTMVTVSS
> h1779_VH.1B
(SEQ ID NO: 77)
EVQLVQSGAEVKKPGASVKVSCKASGYTFTDYIIHWVRQAPGQGLEWIGY
FNPNSGGSNYNENFKRRATMTADKSISTAYMELSRLRSEDTAVYYCARRI
AWDHWYFDFWGQGTMVTVSS
> h1779_VH.1C
(SEQ ID NO: 78)
EVQLVQSGAEVKKPGASVKVSCKASGYTFTDYIIHWVRQAPGQGLEWIGY
FNPNSGGSNYNENFKRRATMTADKSISTAYLEFSRLRSEDTAVYYCARRI
AWDHWYFDFWGQGTMVTVSS
> h1779_VH.1D
(SEQ ID NO: 79)
EVQLVQSGAEVKKPGASVKVSCKASGYTFTDYIIHWVKQAPGQGLEWIGY
FNPNSGGSNYNENFKRKATMTADKSISTAYLEFSRLRSEDTAVYYCARRI
AWDHWYFDFWGQGTMVTVSS

Each of the above light chain variable regions was combined with light chain constant region as set forth in SEQ ID NO: 53 to form the final intact light chain sequences. Each heavy chain variable region was combined with heavy chain constant region as set forth in SEQ ID NO: 52 to form the final heavy chain sequences.

Meanwhile, antibody Hu39D10 against IL5 disclosed in WO2012083370A1 was used as a positive control in the present disclosure, and the heavy and light chain sequence thereof is shown in SEQ ID NO: 80 and SEQ ID NO: 81, respectively.

Heavy chain sequence of Hu39D10
SEQ ID NO: 80
EVQLVESGGGLVQPGGSLRLSCAVSGLSLTSNSVNWIRQAPGKGLEWVGL
IWSNGDTDYNSAIKSRFTISRDTSKSTVYLQMNSLRAEDTAVYYCAREYY
GYFDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
Light chain sequence of Hu39D10
SEQ ID NO: 81
DIQMTQSPSSLSASVGDRVTITCLASEGISSYLAWYQQKPGKAPKLLIYG
ANSLQTGVPSRFSGSGSATDYTLTISSLQPEDFATYYCQQSYKFPNTFGQ
GTKVEVKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Example 6. Preparation of Recombinant Chimeric Antibodies and Humanized Antibodies

### 1.Molecular Cloning of Recombinant Chimeric Antibodies

The positive antibody molecules obtained by hybridoma screening were sequenced to obtain gene sequences encoding the variable regions. Forward and reverse primers were designed on the basis of the obtained sequences, and VH/VK gene fragment of each antibody was constructed via PCR by using the gene sequence as a template. The VH/VK gene fragment was then subjected to homologous recombination with expression vector pHr (comprising a signal peptide and hIgG1/hkappa constant region gene (CH1-Fc/CL) fragment) to construct a recombinant chimeric antibody full-length expression plasmid VH-CH1-Fc-pHr/VL-CL-pHr to form five chimeric antibodies of Ch1705, Ch1706, Ch1780, Ch1773 and Ch1779.

### 2. Molecular Cloning of Humanized Antibodies

Codon-optimization was performed on the designed humanized antibody sequences to generate coding gene sequences with human codon-preference. PCR primers were designed to construct the VH/VK gene fragment of each antibody, and then the VH/VK gene fragment was subjected to homologous recombination with expression vector pHr (comprising a signal peptide and hIgG1/hkappa constant region gene (CH1-Fc/CL) fragment) to construct humanized antibody full-length expression plasmid VH-CHI-Fc-pHr/VL-CL-pHr.

### 3. Expression and Purification of Recombinant Chimeric Antibodies and Humanized Antibodies

The plasmid expressing the antibody light or heavy chain was transfected into HEK293E cells at a ratio of 1:1.2. After 6 days, the expression supernatants were collected, centrifuged at high speed to remove impurities, and purified by protein A column. The column was rinsed with PBS until the A280 reading was dropped to the baseline. The protein of interest was eluted with acidic elution buffer, pH 3.0-pH 3.5 and neutralized with 1 M Tris-HCl, pH 8.0-9.0. The eluted samples were appropriately concentrated, and further purified by gel chromatography Superdex 200 (GE) pre-equilibrated with PBS to remove the aggregates. The monomer peak was collected and aliquoted for use.

The performance and beneficial effects of the antibodies in present disclosure were verified by the following test methods.

### In Vitro Evaluation of Biological Activity

### Test Example 1: Binding of Murine IL-5 Antibodies to IL-5 of Different Species by Biacore Assay

The affinity between the test murine IL-5 antibodies and human IL-5 was determined by using Biacore T200 (GE) instrument.

Molecules to be tested were affinity-captured by Protein A biosensor chip, and then the antigen (the recombinant human, monkey and murine IL5 prepared in Example 1) was flowed through the surface of the chip, and the reaction signals were detected in real time by using Biacore T200 instrument to obtain binding and dissociation curves. After the dissociation of each cycle of experiment was completed, the biosensor chip was washed and regenerated with glycine-hydrochloric acid regeneration solution (pH 1.5). The data was fitted to the (1:1) Langmuir model by using BIAevaluation version 4.1 GE software, and the affinity values were obtained as shown in Table 13.

TABLE 13
Results of affinity of murine IL-5 antibodies
to IL-5 of different species by BIAcore assay

| | KD (M) | | | | |
|---|---|---|---|---|---|
| antigen | mAb1705 | mAb1706 | mAb1780 | mAb1773 | mAb1779 |
| Human | 7.27E−11 | 3.83E−11 | 8.99E−11 | 1.29E−10 | 4.58E−10 |
| IL-5 | | | | | |
| Monkey | 2.05E−10 | 2.77E−10 | 3.12E−10 | 4.76E−10 | 9.98E−9 |
| IL-5 | | | | | |

This example demonstrates that all of the antibodies mAb 1705, mAb 1706, mAb 1780, mAb 1773 and mAb 1779 of the present disclosure have high affinity to IL-5 of different species (human, monkey).

### Test Example 2: Affinity of Humanized IL-5 Antibodies to IL-5 of Different Species by Biacore Assay

The affinity between the test humanized IL-5 antibodies and human IL-5 was determined by using Biacore T200 (GE) instrument.

Molecules to be tested were affinity-captured by Protein A biosensor chip, and then the antigen (prepared in Example 1) was flowed through the surface of the chip, and the reaction signals were detected in real time by using Biacore T200 instrument to obtain binding and dissociation curves. After the dissociation of each cycle of experiment was completed, the biosensor chip was washed and regenerated with glycine-hydrochloric acid regeneration solution (pH 1.5). The data was fitted to the (1:1) Langmuir model by using BIAevaluation version 4.1 GE software, and the affinity values were obtained as shown in Table 14.

TABLE 14
Results of affinity of humanized IL-5 antibodies
to human IL-5 by BIAcore assay

| antibody | KD(M) | antibody | KD(M) |
|---|---|---|---|
| h1705-003 | 3.35E−09 | h1706-003 | 1.89E−11 |
| h1705-006 | 4.11E−09 | h1706-006 | 1.73E−11 |
| h1705-009 | 4.55E−09 | h1706-009 | 5.45E−11 |
| h1705-004 | 2.14E−11 | h1780-017 | 7.78E−11 |
| h1705-007 | 2.21E−11 | h1773-007 | 2.07E−10 |
| h1705-010 | 2.05E−11 | h1779-014 | 4.12E−10 |
| h1705-005 | 2.16E−11 | | |
| h1705-008 | 3.42E−11 | | |
| h1705-011 | 2.30E−11 | | |

The results show that the humanized IL-5 antibodies still have high affinity to human IL-5 (as for humanized variants of the other murine antibodies, only exemplary data were provided, except for each humanized variant of h1705).

### Test Example 3: Murine IL-5 Antibodies Block the Binding Between IL-5 and IL-5α Receptor by ELISA Assay

To demonstrate the ability of IL-5 antibodies to prevent IL-5 from binding to the extracellular region of recombinantly expressed IL-5α receptor protein, ELISA plate was coated with IL-5 (5 ug/ml in PBS), incubated at 37° C. for 1 hour, the liquid was discarded, then 5% skim milk blocking solution diluted with PBS was added at 200 μl/well, and blocked at 37° C. for 2.5 hours in an incubator. After the blocking was finished, the blocking solution was discarded, the plate was washed 5 times with PBST buffer (PBS containing 0.05% Tween-20, pH 7.4), 25 μl of 10 μg/ml IL-5Rα (in 1% BSA) which was labeled with the biotin labeling kit (Dojindo Chemical, LK03) was added, and then 25 μl of gradient-diluted antibody which competed with IL-5Rα for binding to IL-5 was added, and incubated at 37° C. for 1 hour. After the incubation, the reaction solution in the plate was discarded, the plate was washed 5 times with PBST, Streptavidin-Peroxidase Polymer (Sigma, S2438-250UG) diluted at 1:600 with sample diluting solution was added at 50 μl/well, and incubated at 37° C. for 1 hour. After washing the plate with PBST for 5 times, 50 μl/well TMB chromogenic substrate (KPL, 52-00-03) was added, incubated at room temperature for 3-10 min, and 50 μl/well 1 M H₂SO₄ was added to stop the reaction. Absorbance values were read at 450 nm with NOVOStar microplate reader. The IC50 values for IL-5 antibodies to block the binding between IL-5 and IL-5Rα were calculated. The results are shown in Table 15, both antibodies of the present disclosure can effectively inhibit the binding of IL-5 to its receptor.

TABLE 15
Results of murine IL-5 antibodies to block the
binding between IL-5 and IL-5α receptor by ELISA

| | mAb1705 | mAb1706 |
|---|---|---|
| IC50 (μg/ml) | 0.42 | 0.40 |

### Test Example 4: IL-5 Antibodies Block the Binding Between IL-5 and IL-5 Receptor by FACS

To identify the resulting IL-5 antibodies which can block the IL-5 receptor on cell surface, we constructed recombinant cell line CHOS, which highly expresses both receptors of IL-5Rα/β. This experiment identified that IL-5 antibodies can prevent IL-5 from binding to the recombinant IL-5α/β receptor on the surface of CHOS cell line, respectively.

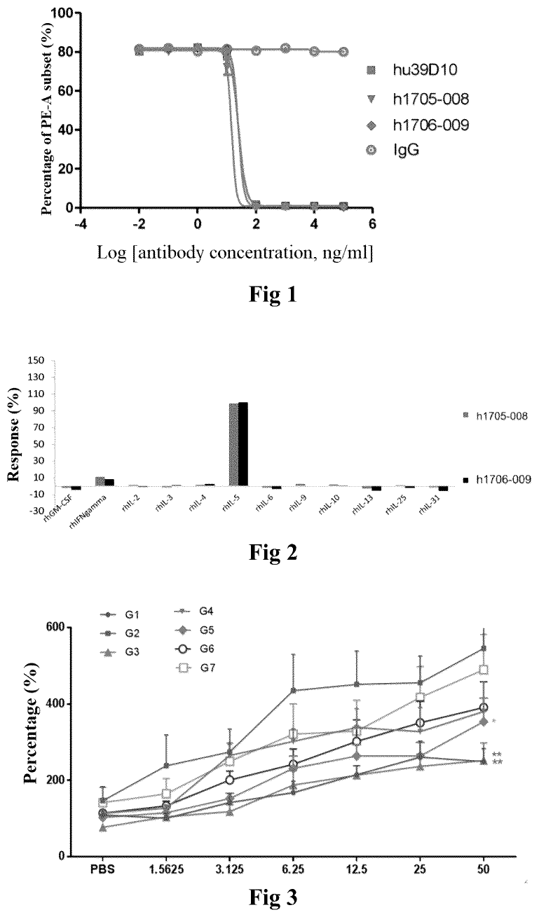

Particular method: CHO-S-IL-5Rα and β were cultivated with CD-CHO containing 100 ng/ml G418 and 25 ng/ml zeozin, and the concentration during cell culture was no more than 3×10⁶ cells/ml. The IL-5Rα/β-CHOS cells in good condition were centrifuged (1000 rpm, 5 min), washed once with 10% FBS in PBS, counted, the cell concentration was adjusted to 4×10⁶ cells/ml, and 25 μl of which was added to 96-well plate with round bottom. Antibodies to be tested were diluted with PBS solution containing 10% FBS, with an initial concentration of 200 μg/ml, and 8 gradients were obtained by 1:10 dilution. 25 μl of 100 ng/ml IL-5 labeled with Biotin Labeling Kit (Dojindo Chemical, LK03), was uniformly mixed with 50 μl of each diluted antibody, added into the 96-well plate to which the cells has been added, and incubated at 4° C. for 1 hour. After the incubation, centrifuged at 4° C. (400 g, 5 min), the supernatant was discarded, centrifuged and washed with 200 μl of pre-cooled PBS, repeat twice; PE-Avidin secondary antibody diluted at 1:1333 was added and incubated in darkness at 40° C. for 40 min, the supernatant was discarded after being centrifuged at 4° C. (400 g, 5 min), 200 μl of pre-cooled PBS was added, the cells were dissociated, centrifuged and washed at 4° C., repeated three times, and 100 μl PBS was added. The plate was read on instrument, and IC50 values for IL-5 antibodies to block the binding between IL-5 and IL-5Rα/B were calculated based on the fluorescence signal values. The results are shown in Table 16 and FIG. 1.

TABLE 16
Results of IL-5 antibodies to block the binding between IL-5 and IL-5Rα/β

| | IgG | hu39D10 | h1705-008 | h1706-009 | h1780-017 | h1773-007 | h1779-014 |
|---|---|---|---|---|---|---|---|
| IC50 | 8777 | 25.07 | 14.51 | 24.74 | 16 | — | 49.64 |
| (ng/ml) | | | | | | | |

The results show that the antibodies h1705-008, h1706-009, h1780-017, h1773-007 and h1779-014 showed stronger ability to block the binding of IL-5 to IL-5 receptor on cell surface.

### Test Example 5: IL-5 Antibodies Inhibit IL-5-Induced Proliferation of TF1 Cells

IL-5 can induce proliferation of TF-1 cells, and IL-5 antibodies can prevent IL-5 from stimulating proliferation of TF-1 cells.

Specifically, TF-1 cells (ATCC, CRL-2003) were cultivated in RPMI1640 containing 10% FBS and 2 ng/mL rhGM-CSF (Lianke Bio, Catalog No. 96-AF-300-03-20), incubated in an incubator at 37° C., 5% CO₂, with the cell density of no more than 1×10⁶ cells/ml. For detection of the antibodies, the cells being in logarithmic growth phase were washed three times with PBS by centrifuging at 800 rpm for 5 min, and the cell density was adjusted to 6000 cells/well/90 μl with RPMI1640 (FBS: 2%, recombinant human IL-5:10 ng/ml). 10 μl of gradient dilutions of antibodies to be tested were added into 96-well plate and cultivated for 3 days. 30 μl of cell titer was added and mixed. The IC50 values were calculated based on the reading. The results are shown in Table 17 below.

TABLE 17
Results of IL-5 humanized antibodies to inhibit
IL-5-induced proliferation of TF1 cells

| antibody | IC50 (nM) | antibody | IC50 (nM) |
|---|---|---|---|
| Hu39D10 | 0.30 | h1706-003 | 0.31 |
| h1705-004 | 0.30 | h1706-004 | 0.30 |
| h1705-005 | 0.30 | h1706-006 | 0.34 |
| h1705-007 | 0.25 | h1706-007 | 0.28 |
| h1705-008 | 0.20 | h1706-009 | 0.25 |
| h1705-010 | 0.30 | h1773-007 | 0.38 |
| h1705-011 | 0.28 | h1780-017 | 0.16 |
| | | h1779-014 | 0.20 |

### Test Example 6: IL5 Antibodies Inhibit IL5-Induced Eosinophil Adhesion

IL5 can induce the differentiation, maturation, migration and activation of eosinophils, cause respiratory inflammation and lead to asthma. According to the principle that IL-5 cytokine can promote eosinophil adhesion and activate eosinophils, in this test example, the effects of IL-5 specific antibodies on blocking the IL-5-mediated pathway were determined by collecting and purifying eosinophils from human peripheral blood, and detecting in vitro the effects of IL-5 antibodies on blocking IL5-mediated eosinophil adhesion.

Specifically, human peripheral blood was 5-fold diluted with PBS containing 2 mM EDTA, monocytes and granulocytes were separated by Percoll™ (with density gradient of 1.088), the erythrocyte layer containing granulocytes was carefully aspirated, and the erythrocytes were removed with Red Blood Cell Lysis Buffer. The remaining cells were counted, Separating Magnetic Beads coated with human CD16 antibody (Miltenyi, Catalog No. 130-045-701) was added, incubated for 30 min and flowed through the magnetic bead column. Effluent of cell fraction was directly collected, which was mainly composed of eosinophils. The resulting eosinophils were counted and added into a 96-well cell culture plate pre-coated with IgG antibody, about 1×10⁴ cells per well, added with human IL-5 (20 ng/ml) and different concentrations of IL-5 antibody molecules (starting at 10 ug/ml, 3-fold diluted, 10 levels of concentrations). The cell culture plate was incubated in an incubator at 37° C., 5% CO₂ for 1 h, and then added with 0.3% CTAB to lyse the cells. Finally, peroxidase reaction substrate TMB was added to develop color. The OD450 absorption values were read under microplate reader. The maximum adsorption value was read out in the well only added with IL-5, and the well containing neither IL-5 nor antibody drug was set as the background control. The inhibition value of each concentration of the antibody drugs relative to the maximum adsorption value was calculated as =(maximum adsorption value−[antibody drug])/(maximum adsorption value−background control value)×100%, and IC50 was calculated. The results are shown in Table 18:

TABLE 18
IL-5 antibodies block IL-5-induced eosinophil adhesion

| | Hu39D10 | h1705-008 | h1706-009 | h1780-017 |
|---|---|---|---|---|
| IC50 | 11.79 | 4.85 | 4.3 | 21.19 |
| (ng/ml) | | | | |

The results indicate that the humanized antibodies of the present disclosure show strong ability to inhibit IL5-mediated eosinophil adhesion.

### Test Example 7: Evaluation of Specificity of Humanized IL-5 Antibodies to Th2 Cytokines

IL-5 is one of Th2 cytokines. To verify that IL-5 antibodies specifically target only IL-5 without cross-reactivity with other cytokines, 12 types of Th2 and related cytokines, including IL2 (R&D, 202-IL-010/CF), IL4 (R&D, 204-IL-050/CF), IL-5 (R&D, 205-IL-025/CF), IFNgamma, IL6 (R&D, 7270-IL-025/CF), IL9 (R&D, 209-IL-010/CF), IL10 (R&D, 217-IL-025/CF), IL13 (R&D,213-ILB-025/CF), IL25 (R&D, 8134-IL-025/CF), IL31 (R&D, 2824-IL-010/CF) and IL3 sharing a receptor with IL-5 (203-IL-050/CF) and GMCSF (R&D, 215-GM-010/CF), were used in Fortebio assay.

Specifically, antibodies were captured by using Protein A Biosensor (PALL Fortebio, 18-5010), the capture signal was recorded, and then 40 nM of each cytokine was added and new binding signals were recorded. Finally, the binding signal for IL-5 was defined as 100%, and the binding signals between antibodies and other cytokines were observed. The results are shown in FIG. 2.

The results show that among the 12 types of the related cytokines, humanized IL-5 antibodies h1705-008 and h1706-009 merely specifically bind to IL-5, and do not show cross-reaction with other Th2 cytokines.

### Pharmacokinetic Evaluation

### Test Example 8: Pharmacokinetic Evaluation of Humanized IL-5 Antibodies in Rats

Experimental SD rats (provided by Sipple-BK Lab Animal Co., Ltd.), 18 males, were divided into 6 groups, with 3 rats in each group. Hu39D10, h1705-008 and h1706-009 were administered intravenously and subcutaneously. Another 9 SD rats were only administered intravenously with h1773-007, h1779-014 or h1780-017. For the intravenous administration group, 0.2 ml of whole blood was collected without introducing anticoagulation before administration and 5 min, 8 h, 1 d, 2 d, 4 d, 7 d, 10 d, 14 d, 21d and 28d after administration. The blood samples were placed at 4° C. for 30 min, centrifuged at 1000 g for 15 min, the supernatants (serum) were placed in EP tubes and stored at −80° C. For the subcutaneous administration group, whole blood was collected before administration and 1 h, 2h, 4 h, 8h, 1 d, 2 d, 4 d, 7 d, 10 d, 14 d, 21d and 28d after administration. The antibody concentration in the serum was determined by Elisa.

The results indicate that the humanized antibody molecules of the present disclosure have long half-life and high bioavailability in rats.

TABLE 19
Pharmacokinetic evaluation of Antibodies in rat

| | Hu39D10 | | h1705-008 | | h1706-009 | | h1773-007 | h1779-014 | h1780-017 |
|---|---|---|---|---|---|---|---|---|---|
| | dosage | | | | | | | | |
| | 5 mg/kg | 5 mg/kg | 5 mg/kg | 5 mg/kg | 5 mg/kg | 5 mg/kg | 5 mg/kg | 5 mg/kg | 5 mg/kg |
| | (IV) | (SC) | (IV) | (SC) | (IV) | (SC) | (IV) | (IV) | (IV) |
| bioavailability | | 68.4% | | 114.3% | | 59.6% | | | |
| T1/2 (day) | 15.1 ± 1.7 | 15.0 ± 4.2 | 15.7 ± 2.3 | 13.3 ± 3.7 | 20.1 ± 4.3 | 17.9 ± 0.5 | 18.2 ± 1.1 | 12.2 ± 0.3 | 14.6 ± 0.8 |

### In Vivo Biological Evaluation

### Test Example 9: Evaluation of Efficacies of IL-5 Antibodies in OVA-Induced Mouse Asthma Model

This test example is to assess efficacies of IL-5 antibodies in ovalbumin (OVA) aerosol-induced BALB/c mouse asthma model based on airway inflammatory response and airway remodeling.

Mice were randomly divided into 7 groups according to body weight, 10 mice in each group: normal control group (G1); model group (G2); treatment groups for antibody h1705-008 (G3 and G4 for two dosages of 10 mpk and 2 mpk, respectively) and h1706-009 (G5 and G6 for two dosages of 10 mpk and 2 mpk, respectively); and a control group for positive antibody Hu39D10 (G7, 10 mpk). On day 1 and day 14, all mice were sensitized by intraperitoneal injection of sensitizing solution. On day 28, 29, and 30, six groups of mice excluding Group 1 were challenged with OVA aerosol challenging solution for 30 minutes. Two hours before the challenge, Group 2 (G2) were intraperitoneally injected with phosphate buffer, and Group 3-7 (G3-G7) mice were intraperitoneally injected with different doses of different antibodies, once a day, for three consecutive days. Fresh antibody solutions to be tested were prepared immediately before each injection and all injections were completed within half an hour. As normal control, 2 hours after intraperitoneal injection of phosphate buffer, the mice in Group 1 were challenged with aerosol PBS for 30 minutes, once a day, for three consecutive days.

On day 31, animals were tested for airway hyperresponsiveness by using WBP system. All animals inhaled spray of methacholine at doubly increasing concentration of 1.5625, 3.125, 6.25, 12.5, 25, 50 mg/mL to determine the enhanced exhalation intermittent value at the corresponding concentration.

On day 31, 1 hour after the test of airway response with WBP system, a trachea cannula with diameter of 1.2 mm was inserted into the trachea and fixed, lung lavage was performed twice, 0.8 ml of phosphate buffer containing 1% BSA and 0.6 mM EDTA was used for each time. The recovered volume of the lavage fluid was recorded.

BALF was centrifuged at 300 g at 4° C. for 5 minutes and the supernatant was retained for analysis of cytokines. After centrifugation, the cells were resuspended in 1.5 ml of PBS (containing 1% BSA and 0.6 mM EDTA) for cell counting. The total number of cells in BALF was counted with hemocytometer and Trypan blue stain assay. The cells were smeared, stained with Wright staining solution for one minute and then stained with Giemsa for 7 minutes to distinguish eosinophils, neutrophils, macrophages and lymphocytes. Cells were counted under optical microscope.

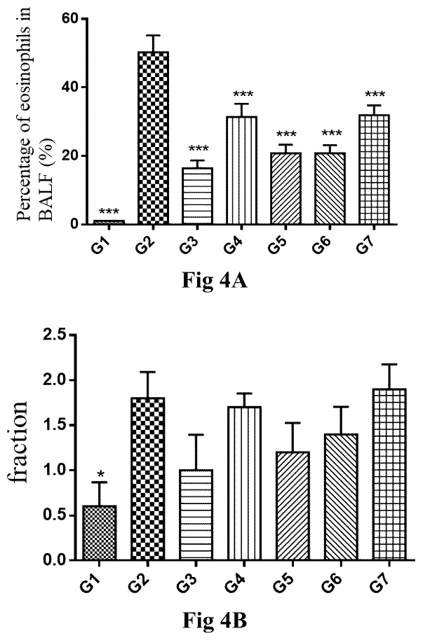

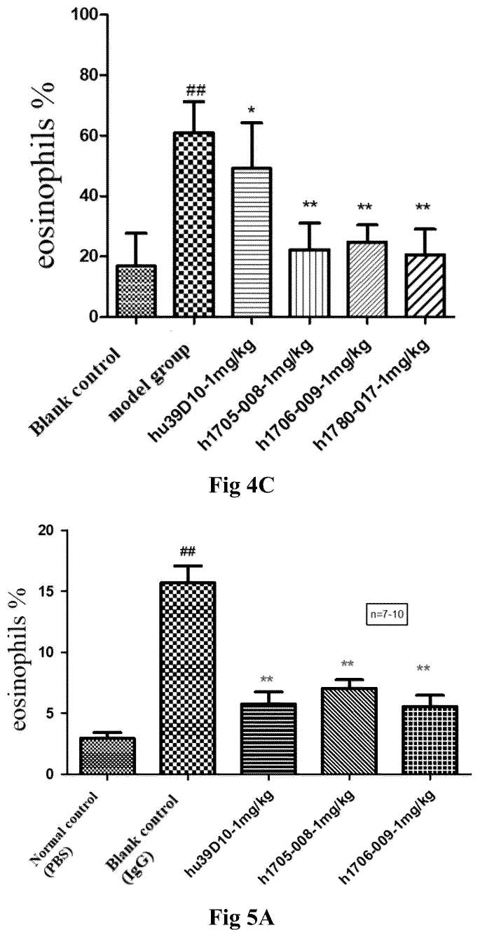

After lavage, lung tissues were collected, immersed in 10% neutral formaldehyde solution and then fixed in 10% neutral formaldehyde solution. The fixed tissues were then subjected to paraffin embedding, trimming, H & E staining, and scoring. The test results are shown in FIG. 3 and FIGS. 4A and 4B.

The results show that the antibody molecules h1705-008 and h1706-009 of the present disclosure can significantly improve lung function in a dose-dependent manner, while high dose (10 mpk) of positive compound cannot improve lung function (see FIG. 3). At the same time, the two antibodies significantly reduced the level of eosinophils and the thickness of mucosa, and exhibited stronger ability to reduce the amount of eosinophils compared to positive antibody at the same dosage (10 mpk) (see FIGS. 4A and 4B). By repeating the experiment in the same type of mouse asthma model, we also verified that 1 mg/ml of h1705-008, h1706-009 and h1780-017 all significantly reduced the level of eosinophils in BalF, when compared with the positive antibody (see FIG. 4C).

### Test Example 10: Evaluation of In Vivo Efficacy of IL5 Antibodies in Exogenous Human IL5-Induced Acute Asthma in Guinea Pig Model

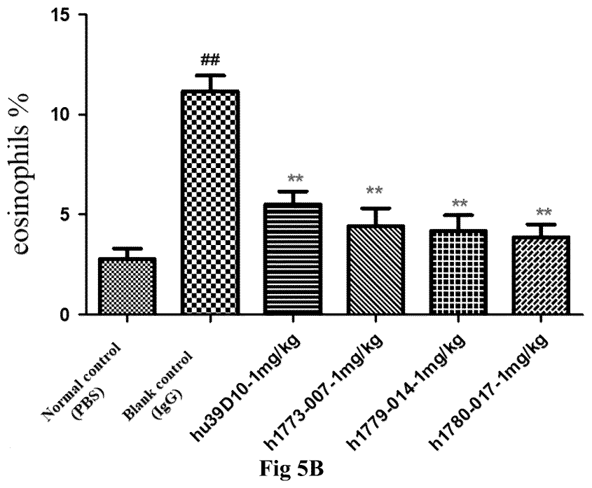

In this test example, male guinea pigs were used to establish human IL5-induced acute asthma model, and hu39D10 was used as a positive antibody to evaluate whether the five humanized IL-5 monoclonal antibodies of the present disclosure have inhibition effects on human IL5-induced increase of eosinophils in Bronchoalveolar Lavage Fluid (BALF) in guinea pig. Guinea pigs were divided into 9 groups, 8-10 in each group: normal control group, model group, hu39D10 (1 mg/kg) group, h1705-008 (1 mg/kg) group, h1706-009 (1 mg/kg) group, h1780-017 (1 mg/kg) group, h1773-007 (1 mg/kg) group and h1779-014(1 mg/kg) group. On day 1, the guinea pigs in the model group and the drug-administration groups were intratracheally injected with 100 μl of human IL5 (containing 5 μg of IL5 antigen) for priming, and the normal control group were intratracheally injected with PBS. 2 hours after the priming, 1 mg/kg of the above IL5 monoclonal antibodies (at the administration volume of 5 ml/kg) were intraperitoneally administered to the drug-administration groups, the corresponding IgG antibody was administered to the model group, and PBS was intraperitoneally administered to the normal control group. 24 hours after intratracheal injection, the guinea pigs were anesthetized and bronchoalveolar lavage fluid was extracted. The cell concentration was adjusted to 5\hat{ }10⁶/ml, and 15 μl of the same was dropped on the glass slide, dried and fixed for HE staining, and the total number of cells and the number of eosinophils were counted under 400× microscope. The percentage of eosinophils was calculated. The results are shown in FIGS. 5A and 5B, indicating that all of the five humanized antibodies of the present disclosure can significantly reduce the level of eosinophils in BALF.

### Evaluation of Stability

### Test Example 11: Stability of Humanized Anti-IL-5 Antibodies

### 1. Chemical Stability of Antibodies

Deamidation modification is a common chemical modification in antibodies which may affect the long-term stability. In particular, high degree of deamidation, oxidation or isomerization modification of partial amino acids in the CDR regions are generally to be avoided, or to be reduced by mutation. 100 ug samples taken at various time points were dissolved in 100 μl solution of 0.2 M His-HCl, 8 M Gua-HCl, pH 6.0, added with 3 μl of 0.1 g/mL DTT, incubated in water bath at 50° C. for 1 hour, and then ultrafiltered twice with 0.02 M His-HCl pH 6.0. 3 μl of 0.25 mg/mL trypsin was added, incubated in water bath at 37° C. overnight for enzymatic digestion. LC-MS analysis was performed with Agilent 6530 Q-TOF. The results show that the chemical stability of the humanized antibodies of the present disclosure was good, and the antibodies did not show abnormal modification after the acceleration reaction in 529 buffer system at 40° C. for one month.

### 2. Study on the Degree of Antibody Aggregation Under High Concentration Condition

The stability of the test antibodies was evaluated at high concentrations and different buffer systems and different temperature conditions for one month. The stability was investigated at the concentration of 50 mg/ml, in three buffer systems of PB9, His and 529, at 40° C., 25° C., 4° C. and −80° C. with repeated freezing and thawing. The degree of aggregation was monitored by SEC-HPLC. Waters e2695 chromatograph was used, with the column of Waters Xbridge BEH 200A SEC, and the mobile phase was PBS (pH was adjusted to 6.8 with diluted hydrochloric acid). 50 μg of protein was loaded and isocratic elution was performed at a flow rate of 0.5 mL/min. It was observed that none of the humanized IL-5 antibodies significantly aggregated under high concentration conditions. After 4 weeks of acceleration reaction at 40° C., the purity of antibody monomer was greater than 95% in all three systems.

### 3. Study on the Purity of Antibodies Under High Concentration Conditions

The stability of the test antibodies was evaluated at high concentrations and different buffer systems and different temperature conditions for one month. The stability was investigated at the concentration of 50 mg/ml, in the His system and at 40° C. The purity was monitored by CE-SDS. 100 μg of protein was taken and sample buffer was added to reach 95 μl. For reducing mode analysis, 5 μl of dimercaptoethanol was added; for non-reducing mode analysis, 5 μl of IAA was added. Incubated in water bath at 70° C. for 10 min, centrifuged and the supernatant was loaded. Data was collected and analyzed with Beckman PA800plus electrophoresis apparatus. It was observed that the antibody proteins have good purity stability under high concentration conditions, and after 28 days of acceleration reaction at 40° C., CE-SDS analysis showed that the main peak of the antibodies was only decreased by 2%.

### 4. Detection of the Thermal Stability of Different Antibodies by UNIT

The samples were dissolved in corresponding buffer (PBS buffer), and the concentration of the samples was controlled at about 1 mg/ml. 9 μl was loaded. Parameter setting: starting temperature 20° C.; incubation 0 s; heating rate 0.3° C./min; Plate Hold 5s; termination temperature 95° C. Tm values of the antibodies were detected. The antibodies have high Tm value and display good thermal stability.

The above described invention has been described in detail with the aid of the accompanying drawings and examples. However, the description and examples should not be construed as limiting the scope of the disclosure. The disclosures of all patents and scientific literatures cited herein are expressly incorporated by reference in their entirety.

1. An isolated nucleic acid molecule encoding an anti-IL-5 antibody or antigen-binding fragment thereof, wherein the anti-IL-5 antibody or antigen-binding fragment thereof comprises a heavy chain variable region and a light chain variable region, wherein:
(i) the heavy chain variable region comprises HCDR1, HCDR2 and HCDR3 region as set forth in amino acid sequences of SEQ ID NO: 16, 17 and 18, respectively; and the light chain variable region comprises LCDR1, LCDR2 and LCDR3 region as set forth in amino acid sequences of SEQ ID NO: 19, 20 and 21, respectively; or
(ii) the heavy chain variable region comprises HCDR1, HCDR2 and HCDR3 region as set forth in amino acid sequences of SEQ ID NO: 22, 23 and 24, respectively; and the light chain variable region comprises LCDR1, LCDR2 and LCDR3 region as set forth in amino acid sequences of SEQ ID NO: 25, 26 and 27, respectively; or
(iii) the heavy chain variable region comprises HCDR1, HCDR2 and HCDR3 region as set forth in amino acid sequences of SEQ ID NO: 28, 29 and 30, respectively; and the light chain variable region comprises LCDR1, LCDR2 and LCDR3 region as set forth in amino acid sequences of SEQ ID NO: 31, 32 and 33, respectively; or
(iv) the heavy chain variable region comprises HCDR1, HCDR2 and HCDR3 region as set forth in amino acid sequences of SEQ ID NO: 34, 35 and 36, respectively; and the light chain variable region comprises LCDR1, LCDR2 and LCDR3 region as set forth in amino acid sequences of SEQ ID NO: 37, 38, and 39, respectively; or
(v) the heavy chain variable region comprises HCDR1, HCDR2 and HCDR3 region as set forth in amino acid sequences of SEQ ID NO: 40, 41 and 42, respectively; and the light chain variable region comprises LCDR1, LCDR2 and LCDR3 region as set forth in amino acid sequences of SEQ ID NO: 43, 44 and 45, respectively; or
(vi) the heavy chain variable region comprises HCDR1, HCDR2 and HCDR3 region as set forth in amino acid sequences of SEQ ID NO: 34, 82 and 36, respectively; and the light chain variable region comprises LCDR1, LCDR2 and LCDR3 region as set forth in amino acid sequences of SEQ ID NOs: 37, 38 and 39, respectively.

2. The isolated nucleic acid molecule according to claim 1, wherein the anti-IL-5 antibody or antigen-binding fragment thereof is selected from the group consisting of murine antibody, chimeric antibody, and humanized antibody.

3. The isolated nucleic acid molecule according to claim 2, wherein the humanized antibody comprises a heavy chain variable region of SEQ ID NO: 49, 57, 63, 69 or 75.

4. The isolated nucleic acid molecule according to claim 3, wherein the heavy chain variable region further comprising a back mutation, wherein:
the back mutation is selected from the group consisting of S49T, V93T and K98S, or a combination thereof on the heavy chain variable region of SEQ ID NO: 49, or
the back mutation is selected from the group consisting of S49T, V93T and K98T, or a combination thereof on the heavy chain variable region of SEQ ID NO: 57, or
the back mutation is selected from the group consisting of R38K, M48I, R67K, V68A, M70L, R72V, T74K and L83F, or a combination thereof on the heavy chain variable region of SEQ ID NO:63, or
the back mutation is selected from the group consisting of F29I, R38K, V48I, R72A, T97F on the heavy chain variable region of SEQ ID NO: 69, and N55V on CDR, or a combination thereof, or
the back mutation is selected from the group consisting of R38K, M48I, R67K, V68A, R72A, T74K, M81L, L83F and D89E, or a combination thereof on the heavy chain variable region of SEQ ID NO: 75.

5. The isolated nucleic acid molecule according to claim 4, wherein the humanized antibody comprises a heavy chain variable region of SEQ ID NO:50 or 51, or comprises a heavy chain variable region of SEQ ID NO: 58 or 59, or comprises a heavy chain variable region selected from any one of SEQ ID NO: 64, 65 and 66, or comprises a heavy chain variable region of SEQ ID NO:70 or 71, or comprises a heavy chain variable region selected from any one of SEQ ID NO: 76, 77, 78 and 79.

6. The isolated nucleic acid molecule according to claim 2, wherein the humanized antibody comprises a light chain variable region of SEQ ID NO: 46, 54, 60, 67 or 72.

7. The isolated nucleic acid molecule according to claim 6, wherein the light chain variable region further comprising a back mutation, wherein:
the back mutation is selected from the group consisting of A43S, L47V, G66R, T69S, F71Y and Y87F or a combination thereof on the light chain variable region of SEQ ID NO: 46; or
the back mutation is selected from the group consisting of A43S, L47M, F71Y and Y87F or a combination thereof on the light chain variable region of SEQ ID NO: 54; or
the back mutation is selected from the group consisting of EID, I2T, 157V, V84T and Y86F or a combination thereof on the light chain variable region of SEQ ID NO: 60; or
the back mutation is selected from the group consisting of M4L, A42S, L45P and L46W or a combination thereof on the light chain variable region of SEQ ID NO: 67; or
the back mutation is selected from the group consisting of A43S, 148V and F71Y or a combination thereof on the light chain variable region of SEQ ID NO:72.

8. The isolated nucleic acid molecule according to claim 7, wherein the humanized antibody comprises a light chain variable region of SEQ ID NO: 47 or 48; or comprises a light chain variable region of SEQ ID NO: 55 or 56; or comprises a light chain variable region of SEQ ID NO: 61 or 62; or comprises a light chain variable region of SEQ ID NO: 68; or comprises a light chain variable region of SEQ ID NO: 73 or 74.

9. The isolated nucleic acid molecule according to claim 1, wherein the anti-IL-5 antibody or antigen-binding fragment thereof comprises:
a heavy chain variable region selected from any one of SEQ ID NO: 49, 50 and 51; and a light chain variable region selected from any one of SEQ ID NO: 46, 47 and 48; or
a heavy chain variable region selected from any one of SEQ ID NOs: 57, 58 and 59; and a light chain variable region selected from any one of SEQ ID NO: 54, 55 and 56; or
a heavy chain variable region selected from any one of SEQ ID NO: 63, 64, 65, and 66; and a light chain variable region selected from any one of SEQ ID NO: 60, 61 and 62; or
a heavy chain variable region selected from any one of SEQ ID NO: 69, 70 and 71; and a light chain variable region selected from any one of SEQ ID NO: 67 and 68; or
a heavy chain variable region selected from any one of SEQ ID NO: 75, 76, 77, 78 and 79; and a light chain variable region selected from any one of SEQ ID NO: 72, 73 and 74.

10. The isolated nucleic acid molecule according to claim 1, wherein the anti-IL-5 antibody is a full-length antibody, and further comprises a human antibody constant region.

11. The isolated nucleic acid molecule according to claim 10, wherein the anti-IL-5 antibody or antigen-binding fragment thereof comprises a human antibody heavy chain constant region as set forth in SEQ ID NO: 52 and a human light chain constant region as set forth in SEQ ID NO:53.

12. The isolated nucleic acid molecule according to claim 1, wherein the antigen-binding fragment is selected from the group consisting of Fab, Fab′, F(ab′)2, single-chain antibody (scFv), dimerized V region (diabody), and disulfide-stabilized V region (dsFv).

13. An isolated nucleic acid molecule encoding an anti-IL-5 antibody or antigen-binding fragment thereof, wherein the anti-IL-5 antibody or antigen-binding fragment thereof comprises a heavy chain variable region and a light chain variable region, wherein the heavy chain variable region comprises HCDR1, HCDR2 and HCDR3 region as set forth in amino acid sequences of SEQ ID NO: 16, 17 and 18, respectively; and the light chain variable region comprises LCDR1, LCDR2 and LCDR3 region as set forth in amino acid sequences of SEQ ID NO: 19, 20 and 21, respectively.

14. The isolated nucleic acid molecule according to claim 13, wherein the anti-IL-5 antibody or antigen-binding fragment thereof comprises a heavy chain variable region of SEQ ID NO: 51 and a light chain variable region of SEQ ID NO: 47.

15. The isolated nucleic acid molecule according to claim 14, wherein the anti-IL-5 antibody or antigen-binding fragment thereof comprises a human antibody heavy chain constant region as set forth in SEQ ID NO: 52 and a human light chain constant region as set forth in SEQ ID NO:53.
